(19) United States
(12) Patent Application Publication
(10) Pub. No.: US 2023/0375583 A1
(43) Pub. Date: Nov. 23, 2023

(54) APPARATUS AND METHOD FOR DETECTING MATTER AND MICRO-ORGANISMS

(71) Applicant: Keith Louis De Santo, St Petersburg, FL (US)

(72) Inventor: Keith Louis De Santo, St Petersburg, FL (US)

(21) Appl. No.: 18/141,374

(22) Filed: Apr. 29, 2023

Related U.S. Application Data
(60) Provisional application No. 63/343,004, filed on May 17, 2022.
(60) Provisional application No. 63/345,825, filed on May 25, 2022.
(60) Provisional application No. 63/353,099, filed on Jun. 17, 2022.
(60) Provisional application No. 63/353,101, filed on Jun. 17, 2022.

(51) Int. Cl. G01N 35/02; G01N 35/00

(52) U.S. Cl. CPC G01N 35/025; G01N 35/00029; G01N 2035/00138

(57) ABSTRACT

A method and apparatus for detecting one or more of a matter and a plurality of microorganisms, comprising: collecting a sample comprising liquid, matter, and/or microorganisms using a plurality of remotely controlled unmanned land, air, and water self-propelled devices; introducing the sample into a sample reservoir connected to a liquid tube inlet; withdrawing the sample from the sample reservoir into the liquid tube having a plurality microscope slides embedded therein through a sample outlet; placing the sample on a sample surface of the plurality of microscope slides; illuminating the sample on the sample surface of the plurality of microscope slides with a light source; intermittently pumping the sample between the microscope slides from the sample reservoir; magnifying the sample on the sample surface of the microscope slides with an image enlargement device; detecting the amount of light transmitted through the sample using a photodetector and/or detecting fluorescence emitted from the sample on the sample surface of the microscope slides using a microscope; analyzing the light detected by the photodetector and/or generating a signal indicative of the fluorescence emitted from the sample on the sample surface of the microscope slides and transferring that signal to a computer software device to determine the presence of matter and microorganisms in the sample; transmitting or displaying the results of the matter and microorganisms detection; controlling the operation of pumps in the liquid tube using a control unit having a plurality of algorithms that manage the operation and identify and classify the sample; detecting a motility and mobility of microorganisms, color of matter, mass of matter, and type of contaminants or matter, wherein the matter and microorganisms is in a singular or combination form; and forecasting a plurality of events in outdoor and indoor environments from data obtained by step l.

### CROSS-REFERENCE TO RELATED APPLICATIONS

This application claims priority to and the benefit of the following: U.S. Provisional Application No. 63/343,004, filed on May 17, 2022, entitled ‘Water Channel Matter Detection’; U.S. Provisional Application No. 63/345,825, filed on May 25, 2022, entitled ‘Detection All Types of Matter by Spinning Balls with Static Electricity, Sticky Substance on Surface or Magnetized Using Artificial Intelligence’; U.S. Provisional Application No. 63/353,099, filed on Jun. 17, 2022, entitled ‘Matter and Micro-organism Universal Detection, Classification and Multipurpose Apparatus’; and U.S. Provisional Application No. 63/353,101, filed on Jun. 17, 2022, entitled ‘Matter and Micro-organism Universal Detection, Classification and Multipurpose Method,’ which are incorporated herein by reference in their entity.

### FIELD OF THE INVENTION

The present invention relates to a liquid tube apparatus for the accurate detection of microbes or matters in real-time. Further, the present disclosure provides an apparatus and method for detecting one or more of a matter and a plurality of micro-organisms in liquid samples conveyed by mobile carriers using computer software programs and machine learning algorithms.

The subject matter discussed in the background section should not be assumed to be prior art merely as a result of its mention in the background section. Similarly, a problem mentioned in the background section or associated with the subject matter of the background section should not be assumed to have been previously recognized in the prior art. The subject matter in the background section merely represents different approaches, which in and of themselves may also be inventions.

### BACKGROUND OF THE INVENTION

Currently, accurate testing before the consumption of food, and liquids and understanding levels of contaminates in living spaces will lower disease in humans, plants, and animals. Municipal utilities have not been able to accurately detect microbes and contaminants in public drinking water, food processing companies have been unable to detect with high accuracy low levels of bacteria and recalls have increased, hospitals and assisted living facilities are breeding grounds for microbes while cleaning companies are making claims that they can eliminate hazardous materials effectively.

The present methods of water purification for distribution through public utilities show false positives and sometimes the impurities are from pipes and retaining basins whereas the data collected is too late and the water has already been consumed. Food processing facilities also have this same problem, hence the increase in recalls over the years. Regardless of what is deemed to be true or false, outsmarting the smartest pathogens and eliminating contaminates has become more expensive, and time-consuming as is not as accurate as they claim to be. The present response times maintain a long duration while machine learning has become the go-to for accurate detection and what response should be taken.

With each passing year, an increasing number of pathogens or contaminants cause illness and fatalities worldwide. The effects of a recent worldwide pandemic continue to generate new variants that further impact populations. Even as of this application filing, polio has been detected in New York wastewater, Legionnaire's Disease was found in the water supply of New Jersey homes, and B. pseudomallei was found in water and soil along the Mississippi Gulf. This marks the first time B. pseudomallei, which can cause the potentially fatal disease melioidosis, has been identified in the United States.

Even with the traditional methods for detecting pathogens, some pathogens may not be detected due to the limitations of these methods, and they are prone to errors and can be expensive to implement, which highlights the importance of detecting and identifying pathogens in a timely, straightforward, low-cost, and accurate manner to prevent the spread of diseases or any harm.

Current methods for detecting pathogens or contaminants in various environments rely on outdated technology and are insufficient for timely disease detection. Some of the commonly used technologies for detecting pathogens include Polymerase Chain Reaction (PCR), Culture-based methods, Next-generation sequencing (NGS), Mass spectrometry, Microarrays, Biosensors, etc. These traditional methods are time-consuming, labor-intensive, and require specialized skills and equipment.

Further, state or municipal workers still manually obtain water and soil samples and wear protective gear to prevent contamination, which can compromise the accuracy of the results. The cost associated with these processes is also a significant concern.

Pathogens are microorganisms that can cause diseases in humans, animals, and plants. They can be found in various environments such as water, food, air, and other environments, posing a significant threat to public health worldwide. Therefore, the detection of pathogens or other contaminants in liquid samples is crucial in various fields.

The present invention relates to an accurate detection of contaminates before the consumption of food, and liquids, in particular to an apparatus and method for detecting one or more of a matter and a plurality of micro-organisms.

Pathogens are microorganisms that can cause diseases in humans, animals, and plants. They can be found in various environments such as water, food, air, and other environments, posing a significant threat to public health worldwide. Therefore, the detection of pathogens or other contaminants in liquid samples is crucial in various fields.

This specification recognizes that there is a need for an apparatus and method that do not use chemicals, require no maintenance, are fully managed by an artificial intelligence and machine learning platform, and provide solutions that leave no residual effect on the environment.

The present disclosure addresses these issues by providing a transparent liquid tube designed for real-time pathogens or contaminants detection in a secure and dependable way. This technology can aid in the detection of pathogens or contaminants, preventing the spread of diseases and reducing the associated costs and risks.

The disadvantages and limitations of traditional approaches will become apparent to the person skilled in the art through a comparison of the described method with some aspects of the present disclosure, as put forward in the remainder of the present application and with reference to the drawings.

### SUMMARY OF INVENTION

An aspect of the present disclosure relates to a method for detecting one or more of a matter and a plurality of micro-organisms. The method includes a step of collecting the liquid, the matter, and the micro-organisms in a plurality of remotely controlled unmanned land, air, and water self-propelled devices. The method includes a step of introducing the liquid, the matter, and the micro-organisms into a sample reservoir connected to a liquid tube inlet. The method includes a step of withdrawing the liquid, the matter, and the micro-organisms into the liquid tube through a sample outlet. The method includes a step of placing the liquid, the matter, and the micro-organisms on the sample surface of a plurality of microscope slides. The method includes a step of illuminating the liquid, the matter, and the micro-organisms on the sample surface of the microscope slides with a light source. The method includes the step of intermittently pumping the liquid, the matter, and the micro-organisms between the microscope slides. The method includes a step of magnifying the liquid, the matter, and the micro-organisms on the sample surface of the microscope slides with an image enlargement device. The method includes a step of detecting the amount of light transmitted through the liquid sample using a photodetector and/or detecting fluorescence emitted from the liquid sample on a sample surface of the microscope slides using a microscope. The method includes a step of analyzing the light detected by the photodetector and/or generating a signal indicative of the fluorescence emitted from the liquid sample on the sample surface of the microscope slides and transferring that signal to a computer software device to determine the presence of matter and micro-organisms in the liquid sample. The method includes a step of transmitting or displaying the results of the matter and micro-organism detection. The method includes a step of controlling the operation of pumps in the liquid tube using a control unit having a plurality of algorithms. The method includes a step of detecting the motility and mobility of micro-organisms, color of matter, mass of matter, and type of contaminate or matter. The matter and micro-organisms can be in singular or combination form. The method includes a step of forecasting a plurality of events in outdoor and indoor environments from data obtained by methods and apparatus operations.

The present invention mainly cures and solves the technical problems existing in the prior art. In response to these problems, the present invention provides an apparatus and method for detecting one or more of a matter and a plurality of micro-organisms.

Another aspect of the present disclosure is to provide a method of detecting pathogens or contaminants in a liquid sample in real-time using the liquid tube as defined herein.

The method may include the steps of: introducing the liquid sample into the sample reservoir; withdrawing the liquid sample from the sample reservoir into the primary liquid tube; placing the liquid sample on the sample surface of the microslides; illuminating the liquid sample on the sample surface of the microslides with the light source; detecting the liquid sample on the sample surface of the microslides with the detection unit; transmitting the image data detected from the detection unit to the control unit; analyzing the data with the control unit to determine the presence of a pathogenic microorganism using machine learning platforms; optionally, transporting the liquid sample to the auxiliary transparent liquid tube for further analysis when the pathogenic micro-organism is detected in the primary liquid tube; and transmitting an alert signal to a user when the pathogenic micro-organism is detected.

Another aspect of the present disclosure is to provide a system for detecting pathogens or contaminants in a liquid sample using the liquid tube as defined herein.

One aspect of the present disclosure is to provide a liquid tube for detecting pathogens or contaminants in real-time. The liquid tube may include a sample reservoir having an entry port and an exit port for holding a liquid sample; a primary liquid tube having a detection unit embedded therein for detecting the presence of pathogens or contaminants in the liquid sample, a sample inlet, and a sample outlet, wherein the sample inlet is connected to an exit port of the sample reservoir; an auxiliary transparent liquid tube connected to the sample outlet of the primary liquid tube, having a detection unit embedded therein for further analysis of the liquid sample feeding from the primary transparent liquid tube; and a control unit in electronic communication with the reservoir, the primary liquid tube, and the auxiliary transparent liquid tube using software programs and machine learning algorithms.

In some embodiments, the liquid tube may further comprise a display unit for displaying the pathogen detection results.

In some embodiments, the liquid tube may further comprise a communication unit for transmitting the pathogen detection results to a remote device such as a smartphone, a tablet, or a laptop or desktop computer

An aspect of the present disclosure relates to an apparatus for detecting one or more of a matter and a plurality of micro-organisms. The apparatus includes a plurality of liquid tubes; a plurality of microscope slides; an oil immersion microscope slide section; a plurality of image enlargement devices; a plurality of remotely controlled unmanned land, air, and water self-propelled devices; a plurality of software program computing systems; a plurality of liquid and air pumps; a plurality of lasers and sensors; and one or more processors. The microscope slides are embedded in the liquid tubes. The oil immersion section may be placed on top of the liquid tubes attached to a plurality of reservoirs. The image enlargement devices are placed on or near the liquid tubes. The image enlargement devices are operated manually, automatically, mechanically, or electronically to enlarge the matter, and the micro-organisms. The remotely controlled unmanned land, air, and water self-propelled devices collect matter and micro-organisms. The software program computing systems utilize software algorithms and software programs written in a plurality of software languages to automatically operate the apparatus and the remotely controlled unmanned land, air, and water self-propelled devices. The software program computing systems direct the elimination of matter and micro-organisms. The liquid and air pumps are controlled by the software program computing systems. The lasers and sensors are controlled by the software program computing systems. The processors execute a plurality of machine learning algorithms and software programs to detect, view and eliminate matter and micro-organisms.

In an aspect, the apparatus includes a plurality of detecting devices and a plurality of computer software programs for detecting matter in real-time.

In an aspect, the matter comprising biological germs, viruses, bacteria, fungus, protozoa, molds, allergens, disease-forming microorganisms (pathogens), non-disease forming micro-organisms (non-pathogenic), microbes, clusters of micro-organisms, a cluster of matter, hydrocarbons, metals, oils, human and animal bodily fluids, plant matter, fertilizers, chemicals, contaminants, and algae in a liquid/wet and/or dry/pseudo-dry liquid tube.

In an aspect, the apparatus includes a plurality of external and internal lights.

In an aspect, the apparatus includes a plurality of light sources emitting light into the liquid tubes and sample reservoirs.

The detection unit may comprise a light source for emitting light into the liquid sample; at least two microscope slides “microslides”, spaced opposite from each other for the liquid sample to travel through the space, each microslide having a sample surface for receiving and holding the liquid sample; and at least one image enlargement device, configured to magnify and detect the liquid sample on the sample surface of the microslides.

In an aspect, the plurality of microscope slides are spaced opposite from each other, wherein both liquid and matter travel through space between the microscope slides, wherein the microscope slides embedded in the liquid tubes maintain a surface for receiving and holding liquid and matter.

In an aspect, the plurality of liquid and air pumps comprises a plurality of processors for monitoring, starting, and stopping the flow of liquid in the liquid tubes using machine learning platforms, algorithms, and computer language software programs.

In an aspect, the apparatus includes a plurality of control units for controlling the operation of the liquid tubes.

In some embodiments, the control unit may be configured to control the intensity and duration of the light source used to illuminate the liquid sample on the sample surface of the microslides.

In an aspect, the microscope slides are adjustable manually or by computer software programs.

In some embodiments, the sample surface of each microscope slide “microslide” may be composed of a material selected from the group consisting of glass, plastic, silicone, and combinations thereof.

The control unit may comprise a processor for operating and managing the liquid tube, and analyzing the liquid sample detected by the detection units to determine the presence of pathogens or contaminants in the liquid sample using machine learning platforms of algorithms.

In an aspect, the image enlargement devices are a plurality of single components and entire light components of a plurality of microscopes.

In an aspect, the microscopes magnify the liquid sample on a sample surface of the microscope slides or between the microscope slides.

In some embodiments, the transparent liquid tube channel may further comprise a photodetector for detecting the light transmitted through the liquid sample on the microslides.

In an aspect, the control units comprise a processor for analyzing the light detected by the photodetectors to determine the presence of the matter in the liquid tubes using a plurality of machine learning platforms, the computer software algorithms, computer software programs, computing software programs, and the proprietary computer language software programs based on the amount of the light detected.

In an aspect, the apparatus includes a plurality of photodetectors for detecting light transmitted through the liquid tubes.

In some embodiments, the image enlargement device may be configured to capture an image of the liquid sample on the sample surface of the microslides to feed the image into the control unit.

The next section is the image enlargement device section used by the apparatus. Many different types of devices and lenses are utilized by the apparatus.

There are many different image enlargement devices the apparatus utilizes. For purposes of understanding the enlargement component device of the apparatus, when the phrase “image enlargement devices are used, it can be any image magnification device listed in this patent application.

An image enlargement device defined for purposes of this patent application is a mechanical or electronic enlargement measuring device with magnification capabilities. The optical lens enlarges the apparent size (the physical size) of matter. Within liquid tubes, more than one image enlargement device may not be needed (they may be focused manually or focused by the algorithms of the apparatus). Image enlargement devices that detect the mobility of micro-organisms, motility of micro-organisms, color, size, and shape that identify objects accurately can also be used by the apparatus.

There are embedded microscope slides into the tube. Image enlargement devices are placed directly above the slides for detection whereas the lenses are connected to computer software and algorithm programs that detect matter in real-time. They may be basic optical lenses, magnification lenses, lenses that are attached to a microscope with a base, folded mirror lenses, light microscopes, electron microscopes, super-resolution microscopes, fluorescent microscopes, x-ray machines, magnetic resonance imaging machines, nuclear magnetic resonance devices, and telescope lenses.

If a specific enlargement device machine is required such as an x-ray machine or magnetic resonance imaging machine, the entire machine will be equipped with a liquid tube running through the machine. Certain methods will be needed such as draining the liquid and the remaining matter is captured on a stage inside the machine where water and liquids will disrupt the application and must be removed first.

Some image enlargement devices will not utilize microscope condensers. Instead of condensers, the section of tube underneath (and close by for surrounding light) the Image enlargement devices will be embedded with lights to illuminate the area between the microscope slides. Some optical lenses each have their very own computer software and algorithm programs that manage all the optical lenses throughout the tube internally and externally. At times when lights embedded on or in the liquid tube are used instead of condensers, computer software, and algorithm programs will manage the brightness of light required for accurate viewing and detection of matter. Image enlargement devices may be placed externally anywhere outside of the tube. The image enlargement devices can be close or far away from the tube. Specific optical magnification lenses may also be placed inside the tube. There is no limit to the amount of image enlargement devices or types of optical magnification lenses or a combination thereof that can be utilized by the UMMDA. Algorithms learn from the data obtained from the Image enlargement devices and optical magnification lenses and transfer that data to other algorithms in the apparatus. The focusing of the image enlargement devices can be manually operated by a single person or with more than one, group of people where each person from the group can manually focus a single image enlargement device. The focusing of the image enlargement devices may also be operated by optical magnification lens focusing algorithms and third-party software programs and can also be automated. The tube component has its very own computer software and algorithm programs that manage the entire tube apparatus.

On certain versions of the apparatus, image enlargement devices may be placed inside the liquid tube in a section that is water tight and located internally inside the tube.

On certain versions of the apparatus, the entire microscope (and its components) may be used. The entire microscope will be defined as all parts that are included when purchasing a microscope from vendors that sell them to the public. The components include but are not limited to the electrical connection, the base, the microscope slide platform, the lenses, the lighting device, and the condenser.

Depending on the length of the tube, there may be many different types of image enlargement devices as listed in this patent application. The image enlargement devices may be located externally outside of the tube or located internally—inside the tube. The image enlargement devices (which include optical magnification lenses and can vary to include folded optics and folded mirror lenses) can be on top of the tube, under the tube, on the side of the liquid tube, or on trusses that brace the liquid tube.

Folded optics is an optical system in which the beam is bent in a way to make the optical path much longer than the size of the system. An example would be prismatic binoculars Prism binoculars have two right-angled glass prisms that apply the principle of total internal reflection. The incident light rays are reflected internally twice giving the viewer a wider field of view. For this reason, prism binoculars are preferred over traditional binoculars.

The microscope slides are located directly below the image enlargement devices. All microscope slides for purposes of this patent application hereafter can be referred to as “slides” or “microslides” and can have different characteristics. For purposes of this patent application, the slides in this patent application will be any type of microscope slides. A microscope slide is defined as a rectangular piece of glass on which samples of matter can be placed for evaluation. The shape of the microscope slides in this patent application can be any shape. Another section below will describe the glass slide component. The thickness of the microscope slides varies from ultra-thin to very thick. The liquid tube usually has two microscope slides with both slides embedded or affixed inside the liquid tube. There also could be a slide topper (externally placed outside on top of the tube) for the oil immersion application. The tube usually has a top slide and a bottom slide with a distance between the two. The tube can be placed horizontally to the ground or vertically to the ground. The design of the tube can have the slides one on top of the other, and in some cases, the slides can be parallel to each other located inside the tube. The distance between the two slides varies. Slides can be made out of any transparent material. Most slides are made of glass.

For applications such as only viewing contaminates, the slides will have a greater distance between them for large singular matter and larger clusters that can be viewed, detected, and identified. If slides are situated in the tube where they are very close together, the application may have filters and screens to only allow very small matter such as singular viruses to flow between the two slides. As discussed below, adjustable slides where the distance between the slides can be adjustable for different applications. An example would be if the apparatus is located in a hospital setting, the distance between the slides would be small to allow only viruses and bacteria to flow through the slides after a filter is placed before the slides to allow only viruses and bacteria to flow between the slides if any are present.

On some occasions, only one microscope slide may be used in each tube. An example of this would be if the manual application is used and the user is viewing larger matter for science projects at a school for dirt and dust particles.

Depending on the application which can range from a small home to a large airport, the size of the job could be millions of gallons of liquid being fed through the liquid tube which may require 5,000 slide sets, or the tube can be equipped with a minimum of one microscope slide or may have more than 150,000 slide combinations.

The apparatus can be equipped with an adjustable microscope slide option for high-end applications for small matter such as viruses. One or two microscope slides can be set in tracks with gears where a tiny mechanical device lowers or rises one or two slides that are on the tracks.

The apparatus can be equipped with three adjustable microscope slides option for high-end identification and detection of several forms of input: large bodies of water, surface matter, and matter that is airborne where the apparatus operates on a higher level.

The apparatus can also utilize the three-stacked slide method. The top slide is on top of a second slide where the top slide is close to the second slide in distance. Another 3rd slide is used in the apparatus where the difference between the second (middle) slide and the 3rd bottom slide is twice the distance.

In an aspect, the plurality of microscopes are configured to provide an image of the liquid sample and the matter sample on the surface of the microscope slides.

In an aspect, the plurality of microscopes are configured to detect fluorescence emitted from liquid and matter on the sample surface of the microscope slides.

In an aspect, an image enlargement device is configured to provide an image of the liquid sample and the matter sample on the surface of the microscope slides.

In an aspect, the image enlargement device is configured to detect fluorescence emitted from liquid and matter on the sample surface of the microscope slides.

In an aspect, the image enlargement device may be a fluorescence microscope.

In some embodiments, the fluorescence microscope may be configured to detect fluorescence emitted from the liquid sample on the sample surface of the microslides or configured to generate a signal indicative of the fluorescence emitted from the liquid sample on the sample surface of the microslides.

In an aspect, the plurality of microscopes are configured to control the intensity and duration of the light source used to illuminate the liquid sample on the sample surface of the microscope slides.

In an aspect, the plurality of microscopes are configured to generate a signal indicative of the fluorescence emitted from the liquid sample and matter sample on the sample surface of the microscope slides.

In an aspect, the plurality of microscopes are configured to capture the image of the matter on the sample surface of the microscope slides.

In an aspect, the image enlargement devices are configured to control the intensity and duration of the light source used to illuminate the liquid sample on the sample surface of the microscope slides.

In an aspect, the image enlargement devices are configured to generate a signal indicative of the fluorescence emitted from the liquid sample and matter sample on the sample surface of the microscope slides.

In an aspect, the plurality of microscopes are configured to capture the image of the matter on the sample surface of the microscope slides.

In an aspect, the image enlargement device is configured to capture the image of the matter on the surface of the microscope slides.

In an aspect, the plurality of image enlargement devices are configured to store the captured image from the surface of the microscope slides in a memory device.

In an aspect, the microscopes are light microscopes.

In an aspect, the plurality of microscopes are super-resolution microscopes.

In some embodiments, the image enlargement device may be a microscope or a laser.

In some embodiments, the image enlargement device may be a folded mirror lens.

In some embodiments, the image enlargement device may be configured to provide the control unit with an image of the liquid sample on the surface of the microslides.

In an aspect, the machine learning algorithms in conjunction with the computing software programs and computer software programs determine a plurality of operations of the apparatus and learn from the operations.

In an aspect, the apparatus includes a communication unit for transmitting the results of the matter and micro-organism detection to a remote device.

In an aspect, the apparatus includes a display unit for displaying the results of the matter and micro-organism detection.

In an aspect, the battery powers the apparatus and a plurality of components of the apparatus.

In an aspect, the remotely controlled unmanned land, air, and water self-propelled devices comprise a plurality of mechanical arms to collect, deposit, move, retrieve, and transport matter and micro-organisms.

In an aspect, the mechanical arms are static, mobile, adjustable, and movable, with pinchers.

In an aspect, the apparatus includes a liquid with matter sample collection receptacle “sample reservoir” for collecting the liquid sample with the matter sample.

In an aspect, the remotely controlled unmanned land, air, and water self-propelled devices comprise a plurality of mechanical arms to deposit matter into a sample reservoir connected to a liquid tube.

In an aspect, the sample reservoirs are connected to the liquid tubes for holding liquid and matter samples.

In some embodiments, a transparent liquid tube may further comprise a liquid sample reservoir unit for collecting a specific liquid sample.

In an aspect, the apparatus includes a plurality of inlets for introducing liquid and matter into the sample reservoirs “reservoirs” and the liquid tubes.

In an aspect, the apparatus includes a plurality of outlets for extracting liquid and matter from sample reservoirs.

In an aspect, the computer software programs may reroute matter into transparent secondary liquid tubes.

In an aspect, the computer software programs may reroute matter into secondary sample reservoirs.

In an aspect, the computer software programs may reroute matter into reservoirs connected to secondary liquid tubes to break apart matter.

In an aspect, the apparatus includes a plurality of outlets for withdrawing liquid and matter from the sample reservoirs and the liquid tubes.

In an aspect, the remotely controlled unmanned land, air, and water self-propelled devices comprise a plurality of mechanical arms to collect, deposit, move, retrieve, and transport matter and micro-organisms and deposit them into a sample reservoir connected to the liquid tube.

In an aspect, the mechanical arms can be static, mobile (attached to UMMDA mobile vehicles), adjustable, and movable, with pinchers.

In an aspect, the apparatus includes a plurality of direct wireless charging systems to power the components of the apparatus.

In an aspect, the apparatus includes a plurality of direct wireless charging systems to transfer charge to a plurality of other devices in the apparatus.

In an aspect, the apparatus includes a plurality of power devices comprising a battery, nuclear power, natural gas, gasoline, and diesel combustible engines, water wheel power (hydro-electric), solar panels, hydrogen fuel cells, wind turbines, and magnetic energy.

Accordingly, one advantage of the present invention is that it cleans, replaces, and removes matter from the platform comprising contemporary and nano-technology sizes, or a combination of both whereas the components of the apparatus are powered by direct wireless charging systems that are further charged by a battery, nuclear power, natural gas, gasoline and diesel combustible engines hydrogen fuel cells, hydrogen fuel cells water wheel power, solar panels, wind turbines, and magnetic energy.

In an aspect, the liquid tubes are connected to a conveyor belt partly submerged in water.

In some embodiments, the control unit can be configured to identify and classify the liquid sample to transmit an alert signal to a user when a pathogenic microorganism is detected.

In some embodiments, the auxiliary transparent liquid tube can be operated when a pathogenic microorganism is initially detected through the primary transparent liquid tube.

In some embodiments, the method may further comprise the step of eliminating the pathogenic microorganism using a biosurfactant. The biosurfactant may be selected from the group consisting of surfactin, iturin, fengycin, lichenysin, serrawettin, phospholipids, rhamnolipid, sophorolipid, trehalolipid, mannosylerythritol-lipids, cellobiolipids, lipoproteins, rubiwettins, trehalose, ornithin, pentasaccharide lipids, viscosin, bacitracin, lipopeptides, and combinations thereof. For purposes of this patent application a biosurfactant will be defined as a chemical secreted from bacteria that whereby the chemical is part of the method of determining how threatening the matter detected in the apparatus is to people, plants, and animals and if the matter can be broken down, altered, or eliminated. The biosurfactant also adds a cleaning, emulsifying agent and to method to dismantle matter clusters in the apparatus components. The components can be mobile, static or both.

The present disclosure provides a highly accurate and efficient means for detecting pathogens or contaminants in liquid samples and can be used in various settings or open and closed environments, including medical facilities, research laboratories, and environmental testing facilities.

In some embodiments, the transparent liquid may further comprise a battery for powering the liquid tube apparatus.

Yet other objects and advantages of the present invention will become readily apparent to those skilled in the art following the detailed description, wherein the preferred embodiments of the invention are shown and described, simply by way of illustration of the best mode contemplated herein for carrying out the invention. As we realized, the invention is capable of other and different embodiments, and its several details are capable of modifications in various obvious respects, all without departing from the invention. Accordingly, the figures and description thereof are to be regarded as illustrative in nature, and not as restrictive.

Other features of embodiments of the present disclosure will be apparent from the accompanying figures and from the detailed description that follows.

In an aspect, the battery powers the apparatus and a plurality of components of the apparatus.

Other features of embodiments of the present disclosure are its power supply whereby the components are powered by singular power generating components or a combination of the following. Electricity, Battery power—power generated from a device in which chemical energy is directly converted to electrical energy, hydro-electric power, magnetic energy, hydrogen fuel cells—power generated from electrochemical cells that convert the chemical energy of a fuel and an oxidizing agent into electricity through a pair of redox reactions. Thermal power—power generation consisting of steam power created by burning oil, liquid natural gas, coal, and other substances to rotate generators and create electricity. Hydrogen power—from a thermal process (natural gas) and electrodes and electrolyte (anodes and cathodes), Solar power—power generated by converting sunlight into electrical energy either through photovoltaic (PV) panels or through mirrors that concentrate solar radiation. Fossil fuels (coal, oil, natural gas)—power generated by fuels that are found in the Earth's crust and contain carbon and hydrogen, which can be burned for energy. Hydro power—power generated by the use of falling or fast-running water. Wind power—power generated from wind by collecting and converting the kinetic energy that wind produces. Both types of nuclear power are provided by fission and fusion. Fusion power—power generated from the heat of nuclear fusion reactions that combine atomic nuclei. Fission power—power generated from the heat of reactions in which the nucleus of an atom splits into two, or more, smaller nuclei. Power generation materials listed above are provided to apparatuses that convert materials into kinetic energy that include turbines, water wheels, and generators. Thrust power can also be used to convert into kinetic energy. Combustion engines are used to convert materials into kinetic energy. The power generated can be stored in power storage devices.

The subject matter discussed in the background section should not be assumed to be prior art merely as a result of its mention in the background section. Similarly, a problem mentioned in the background section or associated with the subject matter of the background section should not be assumed to have been previously recognized in the prior art. The subject matter in the background section merely represents different approaches, which in and of themselves may also be inventions.

### BRIEF DESCRIPTION OF FIGURES

In the figures, similar components and/or features may have the same reference label. Further, various components of the same type may be distinguished by following the reference label with a second label that distinguishes among the similar components. If only the first reference label is used in the specification, the description applies to any one of the similar components having the same first reference label irrespective of the second reference label.

Embodiments of the present disclosure will be described herein by way of example with reference to the accompanying figures, in which:

FIG. 1 is a schematic diagram for the sample reservoir in the transparent liquid tube according to an embodiment of the present disclosure;

FIG. 2 is a schematic diagram for the transparent liquid tube according to an embodiment of the present disclosure;

FIGS. 3A to 3F are schematic diagrams for the detection unit of the transparent liquid tube;

FIGS. 4A to 4D are schematic diagrams for another embodiment of the detection unit of the transparent liquid tube;

FIGS. 5A to 5C are schematic diagrams for the liquid sample collection unit of the transparent liquid tube; and

FIGS. 6A to 6C are schematic diagrams for mobile vehicle carriers of the liquid sample collection unit of the transparent liquid tube; and

FIG. 7 illustrates a flowchart of a method for detecting one or more of a matter and a plurality of micro-organisms, in accordance with at least one embodiment.

In the figures, similar components and/or features may have the same reference label. Further, various components of the same type may be distinguished by following the reference label with a second label that distinguishes among the similar components. If only the first reference label is used in the specification, the description applies to any one of the similar components having the same first reference label irrespective of the second reference label.

### DETAILED DESCRIPTION OF THE ILLUSTRATIVE EMBODIMENTS

The following detailed description is made with reference to the accompanying figures.

It is further important to note that the figures included in the present disclosure are not to scale. While the figures are intended to illustrate the key features and functionality of the invention, they are not intended to represent the size or proportion of any various components accurately. Instead, the figures are intended to provide a clear and concise depiction of the invention that will aid in understanding its operation and functionality. It should be understood that the relative sizes and dimensions of any components may differ from what is shown in the figures and that the figures should not be relied upon for precise measurements or scaling. The description provided herein should be consulted for further details regarding the size and dimensions of the invention.

The detailed descriptions of the illustrative embodiments of this invention are listed in the following sections: introduction to an embodiment of the invention, liquid tubes, microscope slides, lighting, sample reservoirs, mobile vehicles for gathering matter, manual application, computer software devices, GPUs/CPUs, computer software programs, algorithms, third party software programs, proprietary software programs, reports, UMMDA chatbots, auxiliary tubes, alerts, biosurfactant testing reservoirs, matter collection methods, lasers, blacklights, power applications, and power supply devices.

The present disclosure is best understood with reference to the detailed figures and description set forth herein. Various embodiments have been discussed with reference to the figures. However, those skilled in the art will readily appreciate that the detailed descriptions provided herein with respect to the figures are merely for explanatory purposes, as the methods and systems may extend beyond the described embodiments. For instance, the teachings presented, and the needs of a particular application may yield multiple alternative and suitable approaches to implement the functionality of any detail described herein. Therefore, any approach may extend beyond certain implementation choices in the following embodiments.

It is important to note that certain aspects of the present disclosure may not be explicitly described herein. However, it is assumed that these aspects follow a common technical knowledge that is widely known to an ordinary person skilled in art. As such, it is not necessary to provide explicit details regarding these aspects in this disclosure. The skilled person would be expected to understand and implement these aspects based on their general knowledge and expertise. The purpose of the present disclosure is to provide a comprehensive and clear description of the invention while acknowledging that certain aspects may be considered implicit to those skilled in the art.

One aspect of the present disclosure is to provide a liquid tube apparatus for detecting pathogens or contaminants in real-time.

Universal Multipurpose Matter Detection Apparatus short for UMMDA or the Apparatus utilizes liquid tubes, sample reservoirs, mobile vehicles, computer software devices, computer software programs, and algorithms to specifically detect in real-time types of matter and microbes from surfaces, water sources, and from the air.

The primary components of the present apparatus are computer software programs, algorithms, and hardware such as computer software devices, drones, robots, watercraft, mechanical arms, pumps, liquid tubes, and sample reservoirs. The purpose of the liquid tube is to suspend matter in a liquid and move that matter between microscope slides embedded in the liquid tube quickly so that image enlargement devices can view, detect, and identify matter in real-time. The apparatus is designed to automatically and intermittently pump the matter through the liquid tube and detect matter quickly and accurately.

The entire apparatus and its methods of operation are designed to be either managed by computer software and algorithm programs or by a user who can operate the apparatus manually. The apparatus utilizes a series of methods and instructions for both hardware and software. Drones, robots, and watercraft gather matter and deposit the matter into a reservoir connected to the liquid tube. The user can also choose to not utilize the drone, robots, and watercraft component of the UMMDA and gather the matter physically themselves for the apparatus and deposit the matter into a reservoir connected to the liquid tube. Pumps in the reservoir pump the liquid from the reservoir into the liquid tube containing the matter that is circulated through the tube and will flow between the two embedded microscope slides located above or below the image enlargement devices. The number of image enlargement devices will vary depending on the length of the tube. The apparatus is connected to laptops that maintain the algorithms whereby cell phones, tablets, desktops, and servers can be connected wirelessly to the laptops for viewing purposes. The algorithms are also designed to learn (machine learning) from the acquired data and forecast events in the future.

Apparatuses and methods are disclosed for detecting one or more of a matter and a plurality of micro-organisms. Embodiments of the present disclosure include various steps, which will be described below. The steps may be performed by hardware components or may be embodied in machine-executable instructions, which may be used to cause a general-purpose or special-purpose processor programmed with the instructions to perform the steps. Alternatively, steps may be performed by a combination of hardware, software, firmware, and/or by human operators.

The liquid tube apparatus comprises a sample reservoir for holding a liquid sample, a liquid tube having a detection unit for detecting the presence of pathogens or contaminants in the liquid sample, and a control unit for controlling the operation of the sample reservoir and the liquid tube using computer software programs and machine learning algorithms.

Embodiments of the present disclosure may be provided as a computer software program product, which may include a machine-readable storage medium tangibly embodying thereon instructions, which may be used to program a computer (or other electronic devices) to perform a process. The machine-readable medium may include, but is not limited to, fixed (hard) drives, magnetic tape, floppy diskettes, optical disks, compact disc read-only memories (CD-ROMs), and magneto-optical disks, semiconductor memories, such as ROMs, PROMs, random access memories (RAMs), programmable read-only memories (PROMs), erasable PROMs (EPROMs), electrically erasable PROMs (EEPROMs), flash memory, magnetic or optical cards, or other types of media/machine-readable medium suitable for storing electronic instructions (e.g., computer programming code, such as software or firmware).

Various methods described herein may be practiced by combining one or more machine-readable storage media containing the code according to the present disclosure with appropriate standard computer hardware to execute the code contained therein. An apparatus for practicing various embodiments of the present disclosure may involve one or more computers (or one or more processors within a single computer) and storage systems containing or having network access to a computer program(s) coded in accordance with various methods described herein, and the method steps of the disclosure could be accomplished by modules, routines, subroutines, or subparts of a computer software program product.

Although the present disclosure has been described with the purpose of detecting one or more of a matter and a plurality of micro-organisms, it should be appreciated that the same has been done merely to illustrate the invention in an exemplary manner and to highlight any other purpose or function for which explained structures or configurations could be used and is covered within the scope of the present disclosure.

The term “machine-readable storage medium” or “computer-readable storage medium” or storage devices includes, but is not limited to, portable or non-portable storage devices, optical storage devices, and various other mediums capable of storing, containing, or carrying instruction(s) and/or data. A machine-readable medium may include a non-transitory medium in which data can be stored, and that does not include carrier waves and/or transitory electronic signals propagating wirelessly or over wired connections. Examples of a non-transitory medium may include but are not limited to, a magnetic disk or tape, optical storage media such as compact disk (CD) or versatile digital disk (DVD), flash memory, memory, or memory devices.

For purposes of this patent application, a computer is defined as a digital electronic machine that can be programmed through computer language written code to carry out sequences of arithmetic or logical operations automatically. In this patent application, a computer will be referred to as a “computer software device”. Computer software programs will be defined as written computer software code that instructs a computer to perform a task through a set of instructions. The computer software programs “software programs” have subcategories that are referred to as software computing programs, algorithms, software algorithms, machine learning algorithms, artificial intelligence, artificial intelligence algorithms, and algorithmic decision-making software programs. Some computer software programs and algorithms are purchased from third parties that are used by the apparatus while proprietary software programs and algorithms are specifically written for the apparatus.

Depending on what the user desires, UMMDA computer software programs are trained to look for all types of matter such as spheres and rods of bacteria. This type of image recognition “computer vision” uses imaging feeds from the image enlargement devices that process the images in real-time that are readable by a computer software device.

A software program specific to image enlargement devices such as electronic magnification devices, light image magnification devices “light microscopes”, magnification devices using fluorescence “fluorescence microscope” and super-resolution microscopy “laser microscope” focuses on singular and clusters of matter whereby each image enlargement device may use a specific software program. The computer software programs work in unison with certain computer software devices. Computer hardware chip processors such as graphics processing units “GPUs”, field programmable gate arrays “FPGAs”, application-specific integrated circuits “ASICs” and computer processing units “CPUs” are used in computer software devices. The computer software programs are written to work with GPUs and CPUs with the former doing most of the computing. The computer software programs are trained to detect all types of matter with high accuracy in real-time and the UMMDA can transmit the results to the user by various methods such as a final printed report, electronic communication, or verbal (ummda chatbot). For purposes of this patent application, a UMMDA chatbot is defined as a computer software program that uses natural language processing “NPL” to understand user questions and provide responses simulating human responses to a UMMDA user regarding AI matter detection and biosurfactant applications.

The UMMDA has many methods and operations that are managed by at least one computer software device and many computer software programs that utilize software algorithms and software programs written in various software languages. Some of the computer software programs used by the UMMDA are purchased from third parties such as lidar software used with the drones and the exclusive software used by image enlargement devices. Lidar for purposes of this patent application is defined as “LIght Detection And Ranging” whereby lasers are used on the UMMDA drones, robots, and watercraft to map out an area in 3-dimensions “3D” before the matter is obtained. The 3D mapped-out area may provide information to the UMMDA where pathogens and contaminates may be hiding and may focus the UMMDA drones, robots, and watercraft on specific areas. UMMDA drones, robots, and watercraft are used with and without mechanical arms to place and also at times remove matter from reservoirs, with and without devices for collecting matter and with and without lidar. They may also be controlled remotely by the UMMDA, controlled by the user manually, and may be unmanned and manned. For purposes of this patent application, the remotely controlled flying devices (drones), land roaming devices (robots), and water roaming devices (watercraft) will altogether be referred to as “UMMDA Mobile Vehicles”.

The software programs are divided into two sections, software programs and algorithms that operate and manage the UMMDA and specific algorithms that are exclusive software programs that are trained and or have learned to do a specific job. In this patent application, machine learning, artificial Intelligence, computer software programs, and algorithms are all used by the apparatus and the methods of the apparatus and will be referred to as algorithms. The computer software written code language for the algorithms is both off the self (purchased from a third party) and proprietary whereby the algorithms are developed and written, especially for the apparatus and its methods. The most widely used software language for constructing algorithms used by the apparatus is Python while many other software languages may also be used.

The UMMDA also utilizes proprietary software written code that is exclusive to the operation and methods of the UMMDA such as the detection of singular and different types of clusters of matter. Proprietary written language software code was written for various apparatus applications. A specific set of algorithms was written to enable the apparatus to be trained to use a set of rules, problem-solve, learn, and forecast events. Those UMMDA algorithms are numerous.

The apparatus of the present invention acts as a universal multipurpose matter detection apparatus (UMMDA) that detects and views various types of matter from the air, gathered from surfaces and liquid sources.

The apparatus of the present invention acts as a universal multipurpose matter detection apparatus (UMMDA) with many different methods of operation.

The UMMDA detects matter by UMMDA mobile vehicles depositing matter into a sample reservoir connected to a liquid tube where image enlargement devices that interface with the liquid tube create imaging feeds to computer software programs and machine learning algorithms.

UMMDA mobile vehicles for purposes of this invention are defined as remotely controlled flying devices (drones), the land roaming devices (robots), and the water roaming devices (watercraft) will altogether be referred to as “UMMDA Mobile Vehicles”. The UMMDA vehicles can be powered by the different power devices and applications listed in this patent application.

The basic methods and operation of the UMMDA are the gathering of samples of matter from an indoor or outdoor environment utilizing UMMDA mobile vehicles whether the matter is from the air, a surface, or from a water source, whereby the UMMDA can provide a full picture to a user of the UMMDA of what lurks among us humans, plants, and animals.

The UMMDA has both hardware components and software components. The basic hardware components of the apparatus are a computer software device, storage devices, liquid tubes, embedded microscope slides in the liquid tube, mechanically or electronically measuring devices with magnification capabilities “image enlargement devices”, a sample reservoir connected to the liquid tube to deposit liquid and matter into, pumps and computer software programs referred to as computer software programs and algorithms. The basic mobile hardware components are drones, robots, watercraft, and mechanical arms—UMMDA mobile vehicles.

The UMMDA operates in real time where a person “a user”, a drone, a robot, or watercraft (UMMDA mobile vehicles) collects all types of matter using specific UMMDA methods and deposits that matter in a reservoir that is connected to a liquid tube. Embedded microscope slides and slide toppers are positioned above and directly below the image enlargement devices. A pump managed by a UMMDA software program pumps matter suspended in liquid from a reservoir through the liquid tube and through the space between the microscope slides that are directly above and directly under the image-enlarging devices. The pump is managed by a software program that is programmed to pump liquid at certain time intervals so that the image enlargement devices have time to focus on the matter suspended in liquid (if any) between the microscope slides. After focusing, if the UMMDA pump software program does not detect any matter, the pump restarts, and more liquid is pumped between the microscope slides (replacing the prior liquid) whereby possible matter may be detected. This operation is continuous whereby liquid and matter are pumped through the microscope slides and if the algorithms detect something, the algorithms are programmed to place a colored box around the matter. Computer software language programs and algorithms are trained to detect all types of matter from the imaging feeds whereby many different algorithms and software language programs work in conjunction with each other that operate all aspects and components of the UMMDA.

The UMMDA can be operated in two ways by a user. For purposes of this patent application, a user can be a single person, a group of people, or an entity such as a hospital. The user can choose to view matter that the user obtains manually and by depositing matter directly into the sample reservoir or the UMMDA can be fully automatic and obtain and detect the matter by just turning on a switch on the apparatus and have the UMMDA mobile vehicles obtain liquid and matter for deposit into the reservoir.

Liquid Tube is defined as a watertight tube that has embedded microscope slides that pass liquid and matter between the slides. The tube may primarily be made of transparent plastic and glass materials, which can be specifically chosen for their ability to retain liquids and gases mixed with/without matter. However, the tube is not restricted to these materials alone; it may also incorporate a wide variety of alternatives, such as transparent or non-transparent plastics, metal, glass, rubbers, plexiglass, silicone, PVC, ceramic, wood, or any other material conducive to containing liquids. This flexibility in material selection allows for the tube's adaptability to various applications and environments, accommodating factors such as extreme temperatures or specific uses. Therefore, the tube's thickness can be tailored to suit the demands of its intended purpose and environmental conditions, ranging from exceptionally thin to substantially thick, thereby optimizing its performance and durability.

### Methods, Steps, and Explanations of the Invention.

The UMMDA also referred to as the “Apparatus” can be used as a closed system where liquid and matter are continuously circled through the apparatus where only new matter is entered via the reservoir. With this closed UMMDA option, contaminates and microbes detected are specific to an area where the matter was obtained. This closed method is used in closed areas such as a hospital where the target area is confined. A pathogen may be detected in an operating room whereby the staff will be alerted by the UMMDA to contain the pathogen by an alert system such as blinking lights. The UMMDA can also be an open system whereby a pump continuously pumps in new matter and liquid from a large water source such as a lake directly into the liquid tube bypassing the reservoir. This open UMMDA method is used in outdoor areas applications such as a beach where the red tide may be forecasted by the UMMDA. For purposes of this patent application, the matter is defined as anything that has mass and takes up space which includes but is not limited to: biological germs, viruses, bacteria, fungus, protozoa, molds, allergens, disease-forming microorganisms (pathogens), non-disease forming micro-organisms (non-pathogenic), clusters of micro-organisms, hydrocarbons, metals, oils, human and animal bodily fluids, fertilizers, chemicals, contaminants, algae, water, vapors, fluids and solids which broken apart by a blender in the reservoir that may continually turn when the apparatus is on.

As each year turns to the next, more and more pathogens and contaminants are causing death and sickness in people, plants, and animals world-wide. Even a recent worldwide pandemic continues to have an impact by creating more variants. This new apparatus and its methods should give the world-wide population peace of mind that machine-derived facts and the data of supporting those facts are available through the worldwide web in real-time. The present apparatus is based on many different components such as both software and hardware. The descriptions of artificial intelligence in the public domain are not specific to the present apparatus whereby artificial intelligence “machine learning” is just a component of the overall methods. The methods of the UMMDA mobile vehicles, liquid tube, image enlargement devices, reservoir, biosurfactants, and pump methods complemented by a version of a manual application are unique and will classify the present apparatus as both hardware and software apparatus.

With this apparatus, the reporting of threats to society won't become influenced by anyone or anything. This new apparatus will provide conclusions that will be verifiable.

The sample reservoir pumps liquids and matters directly into the liquid tube from the top of the reservoir whereby some of the finer matter that is broken apart by the blending teeth located on the bottom of the reservoir is circulated to the top. There are two ways of breaking apart matter. The blender teeth or a whirlpool method created by the blender spinning a the bottom of the reservoir enables some pieces of matter to be released when water flow from the reservoir comes into contact with the matter. Only a small amount of matter is needed to flow between the embedded microscope slides. A pump in the reservoir can be operated by time intervals and by the duration of the entire operation or can shut down and produce a reverse pumping action to flush out large matter caught in the tube. This reservoir is where the user will deposit matter if operating the apparatus manually. UMMDA mobile vehicles also utilize the reservoir to drop, flow, and place the matter into the top of the open reservoir.

The apparatus is autonomous where computer software programs and algorithms direct, instruct, and make real-time decisions to operate the entire hardware components of the apparatus and the method of direction regarding those hardware components. A version of the apparatus is also offered on a manual basis for certain low-budget applications where the autonomous component can vary from a completely manual operation to certain aspects being automated. Price points will determine the level of automation and specific methods.

The apparatus and its method of detection, identification, and viewing of matter and final results for purposes of this patent application hereafter may be referred to as “matter detection results” or “MDRs”.

The high rate of performance and the collection of many data points in the environment equates to accurate collection of data for the apparatus where millions of data points lead to accurate reports of all types of matter including pathogens and contaminants. The apparatus can detect millions of data points by large lengths of the liquid tube and the amount of and type of image enlargement devices equipped with that liquid tube.

The following components and method of operation of the apparatus are listed and described below. The list of apparatus components is not in order of importance. All components of the apparatus whether they be hardware or software, all work together to produce the final result of detection, identification, viewing, and results—“MDRs”. Each component of the apparatus is required to produce UMMDA MDRs. The user can replace some of the physical applications associated with the UMMDA and input certain requests of the UMMDA, but the algorithms and step-by-step operations of the UMMDA are constant.

The apparatus has two essential parts: 1. Hardware components and, 2. Computer language software programs (software programs) and algorithms. The following section describes computer software devices. The computer software devices are the brains of the apparatus and direct the methods and operations which all work together with the computer software programs and algorithms.

The computer software devices operate by CPUs (central processing units- or computing processing devices) and GPUs (Graphics Processing Unit, video Graphic cards, video creating devices) which are defined as computer hardware for this patent application. The CPU and GPU devices of the apparatus operate through a set of programmable instructions and maintain data in electronic form.

UMMDA CPUs and GPUs within the computing processing devices are required for basic apparatus operation whether the user chooses the manual or the automated option. At least one laptop computer that is programmed with UMMDA computer software programs and algorithms must be connected to the UMMDA whereby cell phones, desktop computers, servers, or tablets with a screen can be connected to the UMMDA or laptop wirelessly or by wire for MDRs.

The computer processing unit's functions and methods are: 1. Controlling the transfer of data and instructions among the various components of the apparatus components and computers, 2. Manage all of the computer's units and components, and 3. Reads instructions from memory, interprets them, and directs the computer's operation of the entire apparatus. The apparatus CPUs and GPUs can: perform at a higher speed than that of humans, perform calculations with 90% accuracy, and perform thousands to billions of tasks simultaneously (exascale instructions or 10 to the eighteenth power) when servers are tethered together for large detection applications such as hospital settings where 100s of thousands of square feet are involved and all the data obtained needs to stored.

GPUs are used for high-performance applications involving algorithms where they work in conjunction with CPUs and other processing devices of the computer processing devices.

For purposes of this patent application, viewing screen devices will be the screens of laptops, cell phones, desktop monitors, server monitors, tablets, and glass walls and plates that display data from the apparatus.

The following section describes the apparatus software operational components which all work together with the computer software programs and algorithms.

The computer software programs and algorithms of the apparatus can be off the shelf or proprietary. For purposes of this patent application, off-the-shelf will be defined as software that was downloaded to the apparatus from 3rd parties over the internet for the operation of hardware components or purchased from a store. The proprietary software will be defined as written code in computer languages (usually Python) specifically for the operation of the apparatus and its components. The written computer code operates, instructs, makes decisions, develops methods for the streamlining of operations of the apparatus, and forecasts future events. The proprietary software developed is specifically for use by the apparatus and its components.

For further reference in this patent application, apparatus will mean the apparatus and all the components that are associated with it as listed in this patent application.

The apparatus has two distinct software programs: 1. Operation, instruction, and a systematic approach “Method” to accomplish a result; 2. Algorithms are those that learn from both the operations and data obtained and the forecasting of events.

Both types of software programs (third-party and proprietary UMMDA algorithms) work together, separately from one another, or on occasion not utilized at all.

Computer software program methods and algorithms for the apparatus are: Instructional, general operation and management of apparatus including specific algorithms that learn from operational mistakes and successes and determine the next steps. Algorithms are either management of methods, operations, instruction, and apparatuses, Algorithms that learn from the matter data obtained, or Algorithms that forecast future events in the environment.

The computer software programs and algorithms for the apparatus can be: Purchased from third parties, built from scratch such as line by line of code developed specifically for the instruction, operation, and learning of all facets of the apparatus, or a combination of both computer software programs and specific algorithms. This specific type of computer software programs are numerous in numbers and maintain proprietary gateways for newer software upgrades to be implemented.

There are many types of algorithms and sub-algorithms such as instructional algorithms for the apparatus, decision-making for the entire operation of the apparatus, and machine learning. Algorithms for the apparatus are based on learning from operations of the entire apparatus, learning from decisions that were made from the apparatus, malfunctions, and mistakes, and forecasting “predictive/prediction”.

Software programs and algorithms work together to instruct, manage, monitor, and make decisions regarding the collection of matter utilizing automated methods. Each method may blend into another method or be completely different from other past methods whereby UMMDA may form a new method from past learnings.

The UMMDA has developed a unique method of training the algorithms for detecting matter and the operation of all the separate components such as pumps, UMMDA mobile vehicles, power devices, charging devices, transfer of power-to-power storage devices, direct wireless charging systems, lighting, adjustable microscope slides, black lights, magnets, cantilevers, reservoirs, blenders, mechanical arms, lasers, and image enlargement devices.

The apparatus is trained to detect all specific types and classes of matter. Trained is to be defined as computer code developed to detect specific matter in detail by data, pictures, video, color, motility, mobility, shape, size (circumference, diameter), and weight. Other environmental sensors, cantilevers, and lasers can also be used for detection as described later in this patent application.

Each matter class as just defined must be “trained”. Trained defined for this patent application is inputting data manually into a database managed by the apparatus. Data is defined as pictures, videos, weights, and other types of data that are specific to each type, class, and category of matter. The type, class, and category of the matter are labeled in colors and boxes and listed on viewing devices. An example of other types of matter data would be the motility and mobility of living matter such as that of a paramecium. As more and more data is compiled by the apparatus, the apparatus learns and manually inputting classes will become less and less.

For purposes of the methods in the apparatus, training an algorithm is also defined as manually adding data by hand by computer programmers. The entire apparatus learns from every method aspect and all the data obtained, builds libraries of learned data, and forecasts possible events that may form in environments.

Classes and training algorithms for the apparatus include but are not limited to: all bacteria classes; bacteria singular—spheres, rods, spirals, strings; bacteria colonies—sphere colonies, rod colonies, spiral colonies; all virus classes; virus—rod types, crowns (spike), spheres; virus—colonies; all classes of Pests, Parameciums, Algae, Molds; all classes of Allergens; all classes of contaminates; general singular matter; and general clusters of matter.

The labeling of data is first in singular form, then in cluster form. The data results are in a cluster form or singular form. If the apparatus detects a cluster of various types of matter, the cluster is rerouted to a reservoir to break apart the matter cluster.

The following identification of matter is labeled as such by the apparatus: Level one colored squared boxes around matter (colors of boxes are subject to change for preference of the user) and notated with text next to the box.

When the matter is shown on the viewing screen as a dark blue box, it is a bacteria rod.

When the matter is shown on the viewing screen as a dark blue box, it is a bacteria rod.

When the matter is viewed on the viewing screen as a blue box, it is a spiral.

When the matter is viewed on the viewing screen as a purple box, it is a sphere.

When the matter is viewed on the viewing screen as a yellow box, it is a paramecium.

When the matter is viewed on the viewing screen as a yellow box with a top red line, it is more than 1 paramecium.

When the matter is viewed on the viewing screen as a light green box, it is a rod colony.

When the matter is viewed on the viewing screen as a light blue box, it is a spiral colony.

When data is viewed on the viewing screen as a red box, it is a sphere colony.

The color boxes may be revised to reflect black light and pet excrement. There are hundreds of other color combinations that depict specific matter and colonies, clusters, and combinations of matter

The color boxes may be revised to reflect black light and pet excrement. There are hundreds of other color combinations that depict specific matter and colonies, clusters, and combinations of matter.

In level two, the matter is labeled pink, red, and purple with double layers of boxes. The matter is labeled Alert. For data labeled as “ALERT” see the section “Matter defined as Pathogenic and matter not defined. UMD—Unknown Matter Detection.

The liquid tube components are described as: Apparatus Hardware Liquid Tube—Internal—The flowing of liquids (with matter suspended in the liquid) internally to the liquid tube. This section has two components: Apparatus Components that may be in the tube internally or externally on or close to the tube: Apparatus components internal to the tube may also be affixed externally whereby the component may be attached, and placed a distance away from the tube. Apparatus components can also be both internal (inside the tube) and (outside the tube) external to the tube.

The liquid tube is one of several components of the apparatus that maintains the flow of liquids.

In one embodiment, the UMMDA has 4 components that make up the liquid tube. 1. The tube that exits the reservoir to the pump is called the “reservoir pump tube”. 2. The tube that exits the pump is called the “pump exit tube”. 3. The tube that connects to the pump exit tube that maintains microscope slides and image enlargement devices is called the “liquid viewing tube”. 4. The tube that exits the liquid viewing tube and connects to the reservoir (on some versions, a pump may be between these tubes) is called the reservoir entrance tube.

For purposes of this patent application, the entire tube system (with all the tube components) will be referred to as the “liquid tube”.

The flow rate of liquid throughout the tube is decided by the force of pumps, the diameter and length of the tube, and the temperature inside and outside the tube.

The calculation of liquid tube volume is in gallons (USA). This is done by taking both volumes of liquid in the reservoir and all tube components. This is calculated by taking the length of all tubes times the diameter and adding the volume of liquid in the reservoir. For example—a 50-inch tube length that is 2 inches in diameter has 0.67999841 gallons in the entire UMMDA tube system. A reservoir that is filled to the fill line which is 5 inches in length, 10 inches in width, and 10 inches in height has 2.16450216 US gallons. Add them both and the total volume of water in the entire apparatus is 2.84450057 gallons. Sometimes the pumps may contain air pockets whereby the volume of liquid in the pumps will or will not be calculated and added to the total volume. For purposes of this patent application, the liquid tube is defined as the main UMMDA apparatus whereby other same UMMDAs can be connected by auxiliary tubes and be called secondary UMMDAs.

The tube utilizes, image enlargement devices, microscope slides, pumps, lights, and sample receptacles “reservoirs”. The purpose of the tube is to suspend matter in a liquid and move that matter between microscope slides embedded in the tube quickly so that image enlargement devices can detect matter in real-time.

The tubes can be singular or two or more connected to each other. The length of the tubes can be as small as 1 nanometer or be many miles long. The diameter of the tube can be large or small depending on the specific detection application. For specific contaminate detection, the diameter may usually be large (1 inch or larger) and for viruses and biological germs, the diameter of the tube may be small (up to 1 inch in diameter). Usually, the tube is transparent and made of plastic but may be made of something else if the tubing has already been in place and some viewing and detection may be added. A presently standing tube such as a municipal water main may be revised and fitted with the components listed in this patent application. Some liquid tubes operate in a liquid/wet and/or dry/pseudo-dry liquid tube or simply a dry tube. Dry tubes without liquids are for powers whereby the application is for solids that were blended and transferred to the tube by air currents such as fans.

The tube can be an elongated triangle, an elongated square, or a circular “cylindrical” tube made up of non-transparent materials or transparent clear glass, plastic, rubber, silicone, transparent ceramics, fused quartz, Polystyrene, Polycarbonate, Acrylic (PMMA), Polyethylene (PE), Amorphous Copolyester (PETG), Polyvinyl Chloride (PVC) Liquid Silicone Rubber (LSR), Cyclic Olefin Copolymers (COC), Ionomer Resin, Transparent Polypropylene(PP), Fluorinated Ethylene Propylene (FEP), Styrene Methyl Methacrylate (SMMA), Styrene Acrylonitrile Resin (SAN), Methyl Methacrylate Acrylonitrile Butadiene Styrene, or a combination of all materials included in one tube. The tube may be constructed of many of the above-listed materials or combinations thereof from a 3D Printer. The 3D printer can also construct a tube with embedded slides.

The tube can also go from a large diameter to a small diameter, from a small diameter to a large diameter, and continue that trend for miles. The tube can be nano-sized or large such as a municipal water tube which can be city blocks in length and more than 1 story high. The color of the tube can be clear (transparent) or solid color (non-transparent) whereby the tube can also bend. The tube can be elongated to any length, nano-sized, or connected to other tubes that can stretch for miles whereby the shape can be any shape that allows the flow of liquids and matter through it.

The liquids that are pumped into and out of the tubes include but are not limited to singular liquids, air mixed with liquids, mixtures of one or more combinations of liquids, combinations of liquids and gases, water, mixtures of gases and liquids, and mixtures of liquid and solid matter. One tube can be made of several different materials and components. The tube may be continuous in length “single long tube” or several tubes may be connected. The tube is too dry, pseudo-dry, or semi-dry or has no air or gas present in the tube.

The main materials of the tube can be and usually are transparent plastic tubes and sometimes glass tubes. The materials of the tube may also consist of but are not limited to the following that maintain liquids and gases: transparent plastics, non-transparent plastics, metal, glass, rubbers, plexiglass, silicone, PVC material, ceramic, wood, and any other materials that can be formed to maintain liquids that are either transparent or not transparent. The tube may be very thick or very thin depending on the application and its surrounding environment such as extreme hot or cold temperatures.

A reservoir for purposes of this patent application will be defined as a watertight receptacle that can hold matter and liquids. The purpose of the reservoir “sample reservoir” is to hold liquids, solids, and all types of matter. The reservoir is connected to a liquid tube whereby matter from a user and UMMDA mobile vehicles can be deposited into the reservoir. Most reservoirs maintain blenders internally that can break down solid matter and create a whirlpool in the reservoir. The reservoir has a replaceable filter between the reservoir and the liquid tube. Reservoirs can be opened, closed, or continually left open.

The open reservoirs that do not close off to other external airborne and surface matter are those applications in environments where particular matter from certain areas is not important. For example, in those applications where specific matter such as fertilizer contamination is identified in a field, it is probably also found not that far away from that specific area whereby rain, wind, and water runoff would have displaced the fertilizer.

Reservoirs can close off for a circulating system and are left open for continual systems.

Some UMMDA apparatuses require closing off the system and only requiring matter deposits at certain times and places while other times the UMMDA apparatus requires the system to be open at all times whereby all airborne and surface matter whether deposited by the mobile vehicles or simply floated or entered the reservoir by chance without the aid of a manual or automatic method.

With certain applications, the liquid entering the tube from a reservoir (or the tube itself) may need to be heated or chilled by a cooling or heating device. The chilling or heating can take place internally in the tube, externally surrounding the tube, or upon liquid entering the tube. This may be done by using basic water heating elements such as those in water heaters in the homes by electricity, gas, or solar power that can be used to change the temperature of the liquid in the liquid tube. For some UMMDA applications, the temperature of the liquid entering (or exiting the liquid tube) may need to be heated or chilled for ease of flow through the liquid tube. Upon the liquid exiting the liquid tube (open tube application such as a pond) the liquid can continue to be pumped or flow into another tube or reservoir called the “Biosurfactant Treatment Reservoir” or “Biosurfactant Treatment Tube” whereby biosurfactants are added to the liquid after detection by image enlargement devices. This biosurfactant Treatment reservoir is where the algorithms learn all about biosurfactants.

The UMMDA also utilizes a biosurfactant application that is used to test if some contaminate detected by the UMMDA can be eliminated, made less toxic, the cell walls of viruses can be penetrated or a microbe can be eliminated/altered. If a contaminate is detected by the UMMDA, the contaminate continues through the liquid tube after detection by image enlargement devices whereby all flowing liquids in the liquid tube must go somewhere. After the UMMDA is turned on and the pumps are pumping liquid (and contaminates are detected and suspended in the liquid) through the tube, the liquid in the tube can: flow continuously in a closed system, flow back into a body of water in an open system or, can continue to flow into other tubes and reservoirs by choice of the user.

If the user opts for contamination, pathogen, or microbe elimination, the UMMDA will test the contaminate, pathogen, and or microbe in a separate reservoir that can maintain elimination liquids. Reservoirs connected to the liquid tube can be specific whereby the reservoir can hold different liquids such as biosurfactants, different combinations, and ratios of Biosurfactants and other environmental liquid applications, and different dilutions of such with different temperatures of the liquids. The UMMDA and the image enlargement devices along with trained algorithms can learn what applications with and without biosurfactants eliminate, disrupt, alter, or break down certain contaminates. For purposes of this patent application, contaminants will be defined as toxic matter and micro-organisms in indoor and outdoor environments that are impure and poisonous that infect humans, plants, and animals by contact or association.

The liquid tubes can be connected with a “Biosurfactant Treatment Reservoirs” or with a “Biosurfactant Treatment Tubes” where the contaminate comes into contact with biosurfactants and or other liquid applications through the tube or reservoir. Separate image enlargement devices are set above or below the biosurfactant treatment reservoirs or the biosurfactant treatment tubes whereby the contaminate elimination identity algorithms can determine if the biosurfactants and their mixtures with other liquid environmental application work to eliminate the contaminate by matching the enlarged image of the contaminate before the biosurfactant application and then after the biosurfactant application.

A database was formed with the following criteria: 1. Did biosurfactants affect the contaminate, and what class and type of contaminate was it? 2. Where was the contaminate obtained, indoors, or outdoors, and what were the surroundings as per the laser 3D map by the UMMDA mobile vehicles? 3. What else was detected during the operation, allergens, molds, urine, excrement, metals? 4. Was the biosurfactant a rhamnolipid and if so, what were the exact ratios from mono to di-rhamnolipid? 5. Were the rhamnolipid mixed with other biosurfactants? 6. Where are the biosurfactants mixed with other environmental liquid applications? 7. What were the carriers used with specific contaminates? 8. What were the dilutions used with specific contaminates? 9. What degree of toxicity was left over? If the image enlargement device shows the contaminate was broken down, how many parts of the contaminate were broken down?

UMMDA tests for the right remediation application whereby the measuring the residual effect on the environment and if the biosurfactant in fact can limit the toxic effect of the contaminant on the environment if any. The applicant's prior rhamnolipid biosurfactant patent applications describe in detail many aspects of Rhamnolipid production and applications. The UMMDA utilizes a separate Biosurfactant reservoir that mixes different types of biosurfactants with detected contaminates whereby when some contaminates are detected by the UMMDA, they are passed to another separate reservoir that uses the same detection application but this time after biosurfactants have been mixed with the detected contaminate in the separate biosurfactant reservoir.

The apparatus is designed to automatically pump the matter through the tube and detect and identify matter quickly and accurately where the entire apparatus and its methods of operation are managed by computer software and algorithm programs. The entire apparatus can be programmed to detect and identify matter with a specific percentage of accuracy. The higher the accuracy of the detection and identification setting, the intermittent pump will pump less often with time in between the pump starting to stop being managed by algorithms.

Tubes can be continual (as in one singular tube), connected to (two or more tubes), or interwoven where the tube can snake in and out of another tube. There can also be tubes that are perpendicular to each other, parallel to each other, or set inside one another. The sizes of the tubes and their connectors can vary in length, diameter, and material.

The main purpose of the tube is to hold two microscope slides where one slide is on top of another. On certain viewing and detection applications where the ambient temperature is not near or below freezing, glass tubes can be used where the glass tube can be formed in a section to replace the two microscope slides where there is enough space for the matter to travel through the space between the top of the glass tube and bottom of the glass tube. The more durable form of the liquid tube is a plastic transparent tube. The space between the slides can be nanometers or many inches apart. If the tube is several inches apart, then the application would be that of contaminants. If the space between the slides is small, the application would be that of viruses. There is a high-end apparatus option to increase and decrease the distance between the two microscope slides manually and automatically. See section Microscope Slides—Mechanically adjusted slides.

The contaminates depending on which application type, sometimes travel in clusters where the viewing application is set to detect only larger cluster types of contaminates. The larger class of matter of combination contaminates include but are not limited to fertilizers, asbestos, metals, oil droplets, parasites, and allergens.

At times microscope lenses (or image enlargement devices) are used without the condenser where the bottom illuminating light is replaced by the light that lines the liquid tube. This method that illuminates the matter directly below the lens for detection is called “running tube lighting”. The lights may be DC powered, powered by electricity, fluorescent light, incandescent light (which can also provide heat), light emitting diode, neon light, halogen light, metal halide lamps, high-intensity discharge lamps, low-pressure and high-pressure sodium lamps, strings of lights. The internally placed lights can be water-tight, waterproof light bulbs. The same lights can also be affixed to the outside of the liquid tube for either lighting or just as a heat source. In some higher-end tubes (costs are much higher for these options), lights may be required to be embedded on the sides of one, both microscope slides, or along the inside of the tube itself (internal). Lights may also be added to the outside of a tube (external) that is clear and transparent. Embedded lights internally or placed externally on the tube. Brighter lights may be required for very small matter such as parvovirus (20 nm), certain gaseous molecules, or metallurgical matter and double-layered lights may be needed. Some lights may also be used to heat the tube internally, or externally or heat other components of the apparatus.

The UMMDA is offered with a gate method where if the apparatus detects matter that is a threat to humanity, it automatically will close off the system and open a gate to another auxiliary tube.

This version of the apparatus is of the higher end (biological germ applications) and detects one of the following: Pathogen, Virus, Biological Germ, or, Unknown matter detection “UMD”.

Each tube is equipped with shut-off valves that can be turned manually (on all versions of the apparatus) at each end of the tube.

Then, several methods are triggered by the apparatus. The first thing the apparatus does is stop the pump. There are four ways in which the matter can be held for extraction and re-evaluated or sent to a lab.

Each tube can have its own pumps in certain apparatus versions where the shut-off valve can be turned 90 degrees to allow for the liquid to be pumped in an auxiliary tube. The tube can be removed by hand.

If the apparatus is in alert mode, and the apparatus version is automated, the second action taken (after the pink, purple, and red-light blinks) is to automatically turn the valves and close off the matter. Then the tube is disconnected from the apparatus by robots for transport by drones. If manually operated, the entire apparatus (or just the liquid tube) can be transported to the lab for further evaluation.

Nanomechanical arms that can be affixed to the tube can also be requested by the apparatus to reach in and retrieve the cordoned-off matter for transport in a separate closed reservoir or a drone or robot or watercraft with a closed reservoir for transporting hazardous material.

While the above steps are taken, concurrently another method will be an alert system. With this other alert method, the apparatus is designed to post the information on a closed-to-the-public system for law enforcement, governments, and health care professionals that are located within the area. The apparatus is designed with several options for alerts.

The next step is to send alert emails. Emails can be sent out through the CPUs, information can be printed and sent out by drone, robot, and watercraft or the apparatus has a method of calling entities from a list of pre-arranged contacts from laptops that have wireless service through an internet service provider.

The matter will be defined as “ALERT”, and the matter will be boxed, and a pink and purple, and red light will blink on the viewing screen devices. Purple and pink lights with red stripes will blink on the viewing screen devices, purple lights will blink on the apparatus. If a biological germ or unknown matter is detected, the apparatus will shut itself down, transmit alerts, and close off tubes to contain the matter. The algorithms are designed to turn on a pump in an “auxiliary tube” also known as a “secondary tube” (connected to the apparatus after the liquid viewing tube part) and pump the threat into another liquid viewing tube whereby a separate image enlarging device and microscope slides are used to reevaluate the threat after it is circled through a separate reservoir that holds a cocktail of biosurfactants. After circling through a separate liquid tube UMMDA than the first one (named the second apparatus with the Biosurfactant option or “Ridcrobe”) if the biosurfactant cocktail can break apart the cell wall of the virus, break down the bacteria or break apart the contaminate, the (main “primary”) liquid tube apparatus will be turned back on, and operations will restart.

The final method after a pathogen or germ alert on the apparatus list is the database over the internet. A website https://www.gvn.ai named the Global Virus Network uses artificial intelligence will alert and give access to all governmental and healthcare organizations worldwide about the unknown matter obtained by the apparatus. If needed, GVN will trigger a contact tracer to stop the spread. The apparatus also pinpoints the location of matter through GPS if desired by the user. A cell phone with GPS is always attached to the apparatus and will simply pinpoint the location for the user. Tracking devices are also located on drones and robots for this same purpose.

Attracting metals to the area below the lenses in the tube is the purpose of magnets. For contamination viewing and detection that may involve metals, magnets may be embedded in the tube to attract metals where the magnets will be placed directly below the lenses so the lenses can view the metal particles. The magnets may be placed externally on the tube or internally in the tube. The externally placed magnets may be pulled a short distance from the tube to release the metal particles or dust and the pumps will clear the tube of the metal fragments if any.

Mechanical arms are used to gather, capture, obtain, transport, and or deposit that matter into a UMMDA reservoir. If any matter including but not limited to metals, microbes, pathogens, viruses, or biological germs is required to be removed from the tube, nanomechanical arms will grab the specific matter and place it in a separate reservoir, tube, or shipping container for later evaluation or shipping.

If the matter detected from the apparatus is a threat to living organisms, it may still require further evaluation whereby mechanical arms will secure the matter for further evaluation which can be determined by other sensors such as lasers or cantilevers. If the matter requires further attention evaluation, a separate tube will maintain a higher range of magnification for further evaluation.

The next section is the image enlargement device section used by the apparatus. Many different types of devices and lenses are utilized by the apparatus.

There are many different image enlargement devices the apparatus utilizes. For purposes of understanding the enlargement component device of the apparatus, when the phrase “image enlargement devices are used, it can be any image magnification device listed in this patent application.

An image enlargement device defined for purposes of this patent application is a mechanical or electronic enlargement measuring device with magnification capabilities. The optical lens enlarges the apparent size (the physical size) of matter. Within tubes, depending on the application, more than one image enlargement device may or may not be needed (they may be focused individually, together, manually, or focused by the algorithms of the apparatus). Image enlargement devices that detect movement, details of cell structure, mobility, motility, color, size, and shape that identify objects accurately can also be used by the apparatus.

There are embedded microscope slides in the tube. Image enlargement devices are placed directly above the slides for detection whereas the lenses are connected to computer software and algorithm programs that detect matter in real-time. They may be basic optical lenses, magnification lenses, lenses that are attached to a microscope with a base, folded mirror lenses, light microscopes, electron microscopes, super-resolution microscopes, fluorescent microscopes, x-ray machines, magnetic resonance imaging machines, nuclear magnetic resonance devices, and telescope lenses.

If a specific enlargement device machine is required such as an x-ray machine or magnetic resonance imaging machine, the entire machine will be equipped with a liquid tube running through the machine. Certain methods will be needed such as draining the liquid and the remaining matter is captured on a stage inside the machine where water and liquids will disrupt the application and must be removed first.

Some image enlargement devices will not utilize microscope condensers. Instead of condensers, the section of tube underneath (and close by for surrounding light) the Image enlargement devices will be embedded with lights to illuminate the area between the microscope slides. Some optical lenses each have their very own computer software and algorithm programs that manage all the optical lenses throughout the tube internally and externally. At times when just lights are used instead of condensers, computer software, and algorithm programs will manage the brightness of light required for accurate viewing and detection of matter. Image enlargement devices may be placed externally anywhere outside of the tube. The image enlargement devices can be close or far away from the tube. Specific optical magnification lenses may also be placed inside the tube. There is no limit to the amount of image enlargement devices or types of optical magnification lenses or a combination thereof that can be utilized by the UMMDA. Algorithms learn from the data obtained from the Image enlargement devices and optical magnification lenses and transfer that data to other algorithms in the apparatus. The focusing of the image enlargement devices can be manually operated by a single person or with more than one, group of people where each person from the group can manually focus a single image enlargement device. The focusing of the image enlargement devices may also be operated by optical magnification lens focusing algorithms and third-party software programs and can also be automated. The tube component has its very own computer software and algorithm programs that manage the entire tube apparatus.

On certain versions of the apparatus, the entire microscope (and its components) may be used. The entire microscope will be defined as all parts that are included when purchasing a microscope from vendors that sell them to the public. The components include but are not limited to the electrical connection, the base, the microscope slide platform, the lenses, the lighting device, and the condenser.

Depending on the length of the tube, there may be many different types of image enlargement devices as listed in this patent application. The image enlargement devices may be located externally outside of the tube, or located internally—inside the tube, or far away from the tube. The image enlargement devices (which include optical magnification lenses and can vary to include folded optics and folded mirror lenses) can be on top of the tube, under the tube, on the side of the liquid tube, or on trusses that brace the liquid tube.

Folded optics is an optical system in which the beam is bent in a way to make the optical path much longer than the size of the system. An example would be prismatic binoculars Prism binoculars have two right-angled glass prisms that apply the principle of total internal reflection. The incident light rays are reflected internally twice giving the viewer a wider field of view. For this reason, prism binoculars are preferred over traditional binoculars. A version of this apparatus utilizes only glass microscope slides.

The microscope slides are located directly below the image enlargement devices. All microscope slides for purposes of this patent application hereafter will be referred to as “slides” and can have different characteristics. For purposes of this patent application, the slides in this patent application will be any type of microscope slides. The thickness of the microscope slides varies from ultra-thin to very thick. The liquid tube usually has two microscope slides with both slides embedded or affixed inside the liquid tube. There also could be a slide topper (externally placed outside on top of the tube) for the oil immersion application. The tube usually has a top slide and a bottom slide with a distance between the two. The tube can be placed horizontally to the ground or vertically to the ground. The design of the tube can have the slides one on top of the other, and in some cases, the slides can be parallel to each other located inside the tube. The distance between the two slides varies. Slides can be made out of any transparent material. Most slides are made of glass.

For applications such as only viewing contaminates, the slides will have a greater distance between them for large singular matter and larger clusters that can be viewed, detected, and identified. If slides are situated in the tube where they are very close together, the application may have filters and screens to only allow very small matter such as singular viruses to flow between the two slides. As discussed below, adjustable slides where the distance between the slides can be adjustable for different applications. An example would be if the apparatus is located in a hospital setting, the distance between the slides would be small to allow only viruses and bacteria to flow through the slides after a filter is placed before the slides to allow only viruses and bacteria to flow between the slides if any are present.

On some occasions, only one microscope slide may be used in each tube. An example of this would be if the manual application is used and the user is viewing larger matter for science projects at a school for dirt and dust particles.

The apparatus can be equipped with an adjustable microscope slide option for high-end applications for small matter such as viruses. One or two microscope slides can be set in tracks with gears where a tiny mechanical device lowers, or rises one or two slides that are on the tracks.

The apparatus can also utilize the three-stacked slide method. The top slide is on top of a second slide where the top slide is close to the second slide in distance. Another 3rd slide is used in the apparatus where the difference between the second (middle) slide and the 3rd bottom slide is twice the distance.

A method that the microscope slides can be adjusted whereby the space between the microscope slides in the liquid tube can be increased or decreased manually by a user or electronically by the apparatus. There are two ways the adjustable slides work. One way is only one microscope slide is adjustable which is the bottom slide whereby the top slide in the liquid tube is embedded into the liquid tube. The second way is the layered 3-slide system whereby the top slide is static, and the bottom two slides are adjustable. Another completely different method is whereby the top embedded microscope slide is embedded in the liquid tube (not movable) the bottom slide is adjustable and a topper slide is placed over the top slide on the liquid tube externally (not movable).

The apparatus can be equipped with three adjustable microscope slides option for high-end identification and detection of several forms of input: large bodies of water, surface matter, and matter that is airborne where the apparatus operates on a higher level. The space between the second and third slides can be 1 inch for the large matter to be detected.

The next section is the pump section where pumps intermittently pump liquids with matter into the tube and out of the tube (placed at both ends of the tube).

The tube has pumps that pump liquids into the tube and pump liquids out of the tube. Pumps can also be inside the tube at any point in the tube, placed outside the tube, or at any point affixed to the tube. The pumps can pump liquids, air, matter, or a combination of all. The pumps can be nano-sized or large industrial-scale tubes such as large municipal water pipes. The tube may be connected by other tubes creating a circular flow or the tube may be open at both ends for a continuous flowing of liquids like that of an ocean. The pumps must be managed by the apparatus to allow the time change between intermittent pumping to allow for more time for the matter to stay under the lenses and between the slides for the auto-focusing application to focus. There can be many pumps to move liquids through many parallel tubes with many lenses to get more data with less time when the time to focus on matter becomes an issue.

Gravity may also be used by placing one end of the tube higher than the other end to have liquid or water flow through the tube without the aid of a pump. This method can be used when the apparatus is physically located lower than a body of water such as a man-made lake on a hill where the apparatus is located on a lower area to the lake. If the UMMDA operation is that of the sea where seaweed and clumps of matter are present such as discarded fishing lines, screens, and or levels of screens may be placed before the pumps to filter out larger material. The screens can be placed one after the other where the space between the screen mesh can become smaller. Pumps are also designed to reverse themselves and create a backwash to flush out debris that has become caught in the screens. The pumps may be placed a short distance below the water line to avoid heaver matter that tends to be near the bottom of the sea. If matter from the bottom of a seabed is needed, robots and watercraft with mechanical arms can obtain that matter and deposit it into a reservoir above the surface of the water whereby the apparatus is located.

The next section is the detection of blood, urine, and semen with black lights.

Black lights can be affixed on the UMMDA mobile vehicles whereby a camera also on the UMMDA mobile vehicle can show the user if blood, urine, and semen are present on bedding, floors, rugs, and walls. These images can be transferred to the UMMDA whereby the images can be transferred to the laptop via wire, wirelessly, or uploaded to a command center for further evaluation.

In some embodiments, black lights can be affixed on the present disclosure whereby a camera also located thereon can show the user if blood, urine, and semen are present on bedding, floors, rugs, and walls. These images can be transferred to the control unit whereby the images can be transferred to the laptop via wire, wirelessly, or uploaded to a command center for further evaluation.

The next section explains the method of delivering results from UMMDA.

Results from detection and identification can be delivered to the user in the form of printed results, verbally spoken results (UMMDA Chatbot), electrically sent results (email and text), or viewed on a glass board that can be manipulated as to visually changing the form and level of results and through sign language and codes of lights. The apparatus can also project results on a separate viewing screen such as walls of glass, Dragontrail, or Xensation. Instructions, pictures, videos of data and results, type of data results, and forecast information can also be seen on Zoom or google meet.

All types of results will hereafter be referred to as “UMMDA results”.

More detailed data can be ascertained with any results as to, how long, length, width, weight (if needed) color, motility, mobility, circumference, diameter, spikes, crowns, surface description, and cluster or singular form of matter. Level two of the viewing report can send video and pictures as well as text. Results also include different color fonts notating the level of contamination of matter, language selection, detail of results and data explanations, maturity and age of matter, and time of the scan.

Through the settings panel of the phone, tablet, video/monitor for laptop, server, and or computer connected by wire or wirelessly to the apparatus, communication software programs offer text-to-verbal communication with the apparatus and verbal-to-text communication with the apparatus. The results from the apparatus can be communicated by the apparatus whereby answers and questions (by both the user and the Chatbot). A microphone on any phone, tablet, laptop, server, or desktop will interact with the apparatus through the communication software programs or “UMMDA Chatbot”.

There are 3 ways matter enters the reservoir:

By a user (by hand), by pumps directly in the UMMDA, or by UMMDA mobile vehicles. Before this happens, the matter must be captured, gathered, or obtained using various methods.

Matter can be obtained from bodies of water, from surfaces or matter can be obtained from the air for detection by the UMMDA. Matter includes solids, liquids, and gases. Matter can be gathered manually by a user, gathered through automation by the UMMDA mobile vehicles, or a combination of both. There are 2 ways matter enters the UMMDA. By pumping liquids directly into the UMMDA or by depositing matter into the UMMDA reservoir. If the manual version is chosen (UMMDA mobile vehicles are not used), the user collects matter by hand and deposits the matter into the liquid tube reservoir.

Liquids can have a high density of matter “clusters of matter” throughout the liquid, a low density of matter, or just a singular microbe or several molecules such as over 1.5 sextillion molecules as in a drop of water. The liquid can easily be pumped and easily flow through the liquid tube such as water or be in the form of a thick liquid where contaminates are mixed in the liquid or the liquid is itself thick like ethylene glycol. If the apparatus determines that a liquid is too dense to travel through the tube (pipe flow meters located in the liquid tubes) the pipe flow meter software will shut down the UMMDA. The liquid tube and reservoir will need drained and or cleaned of the liquid. The filter between the reservoir and liquid tube depending on the level of and density of matter may have to be cleaned or changed physically. The apparatus monitors flow through the liquid tube by a pipe flow calculator (flow meter) that determines that the pump or pumps in the liquid tube(s) are under duress. Pressure in the pipe can also be calculated as a sign that the pump(s) are under duress. At times, water may be added to the reservoir and or liquid tube for ease of flow of the liquid. If oil is present, the oil may be diluted with a biosurfactant or a solvent in the reservoir. For purposes of this patent application and this section, matter can be mixed with liquids, liquids can be mixed with matter, and gases and vapers can be mixed with both.

UMMDA manual method—user is required to use their hands holding a cloth to wipe surfaces with that cloth and rinse the cloth in the liquid of the reservoir. The user can also use dabbing and dragging devices such as a small garden rake to obtain surface matter.

Continual and Circulating Method. Two methods of gathering liquids and water. There are two ways of pumping the liquids through the tube to obtain optimum detection of matter. An unlimited amount of liquid enters the system known as the “continual method”. This method is utilized for large bodies of water. A finite amount of liquid is known as the “circulating method” This method is used with the matter being deposited into reservoirs whereby the liquid and matter in the entire system (liquid tubes and reservoirs) are constant.

The matter is obtained from water sources such as streams and brooks (running water) and from large bodies of water such as lakes and oceans. Pumps are placed anywhere water is available. Sizes of pumps can be nanoscale to very large pumps such as those used in municipal water treatment plants. The continual method can be used to have liquids pumped into the reservoir or directly into the UMMDA liquid tube bypassing the reservoir. UMMDA mobile vehicles nor the user are used with continual method or direct pumping into the apparatus from a body of water.

With this method, the reservoir can either be drained and replaced with new liquid from the drones and robots or the reservoir can be left alone to overflow and excess liquids can run over the side of the reservoir. With this method, the apparatus can be located on a slanted hill whereby the excess fluids can run right back into the water source. Or, the fluids can run over the sides into a drain or sewer located nearby.

Water from a body of water such as an ocean can also be pumped into a reservoir equipped with a blender. If a water source (body of water, brook, lake stream, municipal water treatment plant) is located far away, drones and or robots will be needed to fill a tank with water or liquids that may be needed by traveling back and forth carrying the liquids from a tank equipped with a pump to fill the tank.

Liquids are pumped into the liquid tube from a body of water such as a lake or ocean. This method utilizes a static form of operation (no UMMDA mobile vehicles are utilized) where the apparatus sits near on top of a body of water. The liquid tube is open on two ends and pumps are located at the beginning of the liquid tube, in the middle if needed, and at the end of the liquid tube. Most of the time, in large bodies of water many different types of microbes, matter, organic material, and inorganic material flow through the water. With this, pumps can capture all types of matter in those bodies of water and waterways such as streams. The pumps can be set on the water bed, anywhere between the water bed and the surface of the water, or at the surface of just at the shoreline to collect liquid, liquid matter (combination of liquid and matter) and the material on the waterbed hereafter referred to as muck.

To repeat, there are two ways matter enters the UMMDA. By pumping liquids directly into the UMMDA from a large body of water hereafter known as the “UMMDA Continual method” or by depositing matter into the UMMDA reservoir. The continual method as discussed above can be used to have liquids pumped into the reservoir or directly into the UMMDA liquid tube bypassing the reservoir. When the circulating method is used, circulating the exact amount of liquid until either detection of matter has been accomplished or the liquid in the entire system has not shown any matter or new data where no more liquid enters the entire system through the reservoir. When the circulating method is used, the matter detected and class identified creates a log of the same class of matter that is identified and detected more than 10 times. The liquid entering the reservoir can also be dropped/dripped/by use of gravity/placed by a mechanical arm that is static or attached to a UMMDA mobile vehicle. The user may choose to obtain matter and liquids manually whereby depositing the matter and or liquids into the reservoir.

The amount of liquid and matter in the entire system at one time cannot reach a certain level. The purpose of this method is to get a more accurate data set whereby liquid and matter continually circle the system whereby matter in the liquid will most likely be pumped through the space between the microscope slides in the liquid tube. If the UMMDA circulates the liquid for a specific set of time (duration), and the same matter is detected more than 10 times, the apparatus will send a message or talk through the chatbot to the user, or blink in green that apparatus is ready for more liquid and or matter to be deposited into the reservoir. The same type of notification or a half-brown light/half-yellow light means that the system must be drained and or the filter must be replaced in the reservoir. A white blinking light means that the filter must be replaced. This method can have a certain amount. The user or the drones and robots can initiate this method if chosen by placing the tubes in the water randomly or by the UMMDA 3d view whereby the UMMDA algorithms direct where the drones and or robots can place the connecting extension tubes for the pumps to the UMMDA. The algorithms have learned where and where not to place the extension tubes.

Closed systems usually are for pathogen or biological matter in hospital and military and law enforcement applications. The (open and close tight) to external airborne and surface matter are those applications for biological germs, pathogens, and viruses such as hospital settings where many different pathogens lurk in the air and on surfaces. For example, those applications where specific matter obtained from a certain area is important such as specific matter obtained from an emergency room where human traffic is high and needs to be monitored.

As another example, hospitals with severe burn victims cannot be subject to any outside matter. With skin not there to protect from invading pathogens, this particular victim this application may use a swab put into a sterile tube for transport so that damage to the patient and tainting of the matter is limited.

This learning process is continual for the UMMDA. Other methods of collecting matter such as from surfaces and the air are in the next section.

As described in the last section, matter, and liquids can be transferred into the liquid tube apparatus through pumps set in a body of water or deposited into the UMMDA reservoir. The UMMDA can be operated manually or automatically when collecting matter. The manual method requires a physical effort by the user whereby matter is obtained by hand. The automatic method of capturing matter which is hereafter referred to as “automated” utilizes UMMDA mobile vehicles. The UMMDA can be programmed whereby both the automated and the manual methods can be utilized at the same time.

The methods of obtaining and capturing matter to deposit into the liquid tube (sometimes it could be dry inside the tube) are as follows: 1. UMMDA mobile vehicles using woven and screen mesh materials; 2. UMMDA mobile vehicles using columns; 3. UMMDA mobile vehicles dabbing and dragging method with mechanical arms; 4. UMMDA mobile vehicles dabbing and dragging method without mechanical arms; 5. UMMDA mobile vehicles with a sticky substance method with mechanical arms; 6. UMMDA mobile vehicles with a sticky substance method without mechanical arms; 7. Mobile vehicles with mechanical arms for picking up objects; 8. Mobile vehicles spinning fan while charging with DWCS.

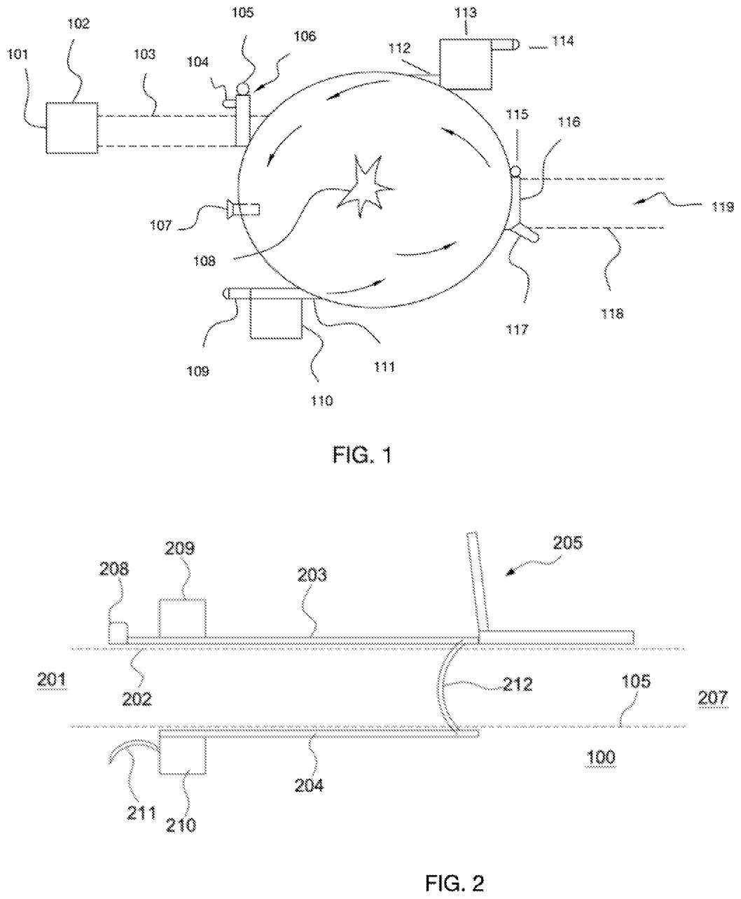

Using these methods, there is only one opening located at the top of the reservoir where UMMDA mobile vehicles deposit, place, unload, dump, or drip a small amount of combined liquids and matter into the reservoir. The FIG. 1 shows “reservoir opening” whereby the reservoir can be opened at the top, semi-opened, or almost fully closed which is connected to a liquid tube. The blenders in the reservoirs and pumps continuously circulate the liquid with the new matter being entered into the system via the reservoir. The liquid can be changed when the software programs determine when the filter is clogged when no filter is present, when circulating liquid becomes saturated with matter that has already been detected or the liquid in the entire system reaches full capacity. Full capacity is reached when the fill line in the reservoir is breached.

The automated application (after mapping out a 3d view of the area) generally obtains the liquids from a large body of water such as a pond in the backyard. Manual applications are not as accurate due to the limited amount of data obtained and the possible tainting of matter by humans. With drones and robots, many samples of matter can be obtained in 24 hours as the drones and robots can keep making matter deposits into the reservoirs, humans can get tired and start to make mistakes by obtaining samples from the same areas where drones and robots are programmed to obtain samples from every part of the body of water. With this method, the reservoir can either be drained and replaced with new liquid from the drones and robots or the reservoir can be left alone to overflow and excess liquids can run over the side of the reservoir. With this method, the apparatus can be located on a slanted hill whereby the excess fluids can run right back into the water source. Or, the fluids can run over the sides into a drain or sewer located nearby.

UMMDA mobile vehicles use woven and screen mesh materials. Woven mesh material for capturing airborne matter with drones, robots, and watercraft. Metal, plastic, and fabric mesh are attached to solid frames in UMMDA mobile vehicles to collect airborne matter. Air flow is created by both movements of vehicles and propeller movement “prop wash” of drones, robots, and airboats. By creating air flow through woven material, the airborne matter will become caught in the small holes in the mesh. For purposes of this patent application, airborne matter is defined as any type of matter floating in the air including microbes and contaminates that may be attached to dust, skin cells, or be a mixture of solid particles and liquid droplets. This airborne matter can be caught in woven materials such as metals, plastic fabrics, or any material that is woven together. The materials woven together can be one or more than one different material. For purposes of this patent application, woven materials means any type of material or different materials that are woven together to form a mesh that is placed in or on a solid structure such as a cylinder or solid frame that is attached to a UMMDA mobile vehicle. This solid frame surrounding the mesh and holding it in place that is attached to a mobile vehicle is used to capture matter that is floating in the air for testing in the UMMDA.

After the capture of some matter from the air, the mesh is flushed with a liquid “rinsed with liquids” while the mesh is over a reservoir connected to the liquid tube. The airborne matter captured in the mesh makes its way with the rinse into the reservoir which then is pumped through the embedded microscope slides for detection. For purposes of this patent application, this process is called UMMDA meshing and rinsing deposit into the reservoir.

Each UMMDA mobile vehicle has a unique method of collecting airborne matter and releasing the collected matter into the UMMDA reservoir connected to the liquid tube.

The drones utilize mesh that is located in a prop tube column to capture matter from the air. The prop wash pulls (or pushes) air through the tube whereby matter floating in the air becomes lodged in the mesh. After drones have completed a pursuit of airborne matter in an assigned area, the drone will recharge its battery by setting itself down in an exact position over the reservoir on a charging pad. This charging pad is connected to the apparatus and supplies power to the pad so it can charge the drone while the mesh in the drone is rinsed. The pad is waterproof and is insulated from the liquids used to rinse the mesh of some (not all) captured matter. Since the drone and columns of mesh are positioned over the direct wireless charging system pad (DWCSP), the rinse with captured matter from the air is transferred (dripped or flowed) into the reservoir with the dripping liquids from the rinse.

The columns on the drones have inlets where streams of water or liquids are sprayed into the inlets from the reservoir. The matter combined with water or liquids is rinsed from the mesh and falls (is transferred) directly into the reservoir.

The prop wash from the propeller(s) draws air through the column whereby dust and matter and matter attached to dust gets caught in the screens. After 15 minutes of flight, the drone will sit on top of the reservoir connected to a liquid tube to drain the columns of matter caught in screens while charging over a pad that charges the battery.

Columns in both drones and robots maintain the woven mesh that is tilted 45 degrees (in the column) which only takes up half of the diameter of the columns. The reason for the positioning of the screens (45 degrees) is to allow for a liquid rinse to have a direct line to flow down through the column into the reservoir connected to the liquid tube. The nozzles from the reservoirs are positioned to stream water or another type of liquid directly into the column portals while the drone is charging.

Airboats can capture airborne matter by placing a screen in front of the propeller For purposes of this patent application, airboats are defined as floating devices with a propeller above the water surface to be used as a propulsion device on water. The woven mesh can be sprayed with a stream of water that drips into a funnel with a tube that flows into the reservoir connected to a liquid tube.

Robots utilize different methods of obtaining airborne matter. With most UMMDA detection applications, drones are used to capture airborne matter for UMMDA meshing and rinsing. On some versions of the UMMDA, drones are not used and only robots are used. Some airborne matter must be tested with this UMMDA robot version. An example of this is an assisted living facility where both staff and the residents will allow robots to roam the floor but flying vehicles will cause stress. With this UMMDA version, the robots utilize fans in columns of screens whereby the airflow through the columns induced by the fans will capture matter on both the mesh and the fan blades. The robot columns are located higher than the rim of the reservoir connected to the liquid tube whereby the robot can sit on a DWCSP and charge while the column is titled at a 45 degree angle. At the top of the column, a water hose can be put in manually to flow water through the column and rinse the matter into the reservoir.

Screens with and without fabrics can be attached to the bottom of drones whereby the screens serve two purposes. They sit below the propellers (props) whereby prop wash and airflow will collect matter. Air both indoors and outdoors usually maintains airborne matter such as dust, allergens, molds, and contaminants. The dust that the screen collects usually has other matter that has attached to the dust. The screens are on, in, or are the landing gear of the UMMDA drones and when placed anywhere on the drone, air traveling through the screens will collect airborne dust.

With the reservoir depositing method of the drone, the opening of the reservoir has rails placed across the top of the reservoir. The screen is small enough to fit through the rails whereby the level of liquid in the reservoir is right below the rails. The screens and matter gathered on the screens are submerged in the reservoir when the drone is resting on the reservoir rails.

The user can replicate this method using a manual application whereby a fan with fabric draped over the fan will eventually collect matter. After turning off the reservoir blender, the user can manually place the fabric in the reservoir (below the water surface) which will release some matter into the reservoir. The user can also choose to wring the fabric out of liquids by hand after placing the fabric below the surface of the water in the reservoir.

Method of matter deposited from a UMMDA mobile vehicle into the top of a reservoir.

By this method, the UMMDA mobile vehicle can deposit matter into the UMMDA reservoir in many ways:

By utilizing a mechanical arm “arm” on a UMMDA mobile vehicle, the arm can place the matter into the reservoir with little to no splashing of the matter,

If splashing is not an issue (detection of allergens is a primary goal of the user whereby allergens are ubiquitous), a drone flying over the reservoir can drop the matter sample into the reservoir whereby some splashing will take place and sometimes misses will happen where wind gusts will cause such an occurrence and the matter is dropped outside of the reservoir, or by dropping the liquid or matter directly onto a conveyor belt whereby the conveyor belt end sits over the top of the reservoir.

The UMMDA method of gathering matter from surfaces. There are 2 ways of gathering matter from surfaces. The UMMDA mobile vehicles perform a wiping method with a cloth. If the user chooses the manual wiping method whereby the user can wipe a surface with fabric and deposit the matter in the same manner as the method of submerging the fabric below the water line in the reservoir. The UMMDA mobile vehicles surface gathering method uses a mechanical arm whereby the mechanical arm holds the fabric in its claw, wipes a surface with fabric, and submerges the fabric below the water surface in the reservoir. The mechanical arm can also use a method whereby a fabric is wrapped around the mechanical arm and touches, wipes, dabs, or drags the fabric along a surface to collect matter in the woven strands of the fabric and the space between them.

Drones as a mobile component to the UMMDA have different methods of operation: Map a 3D view of the area that UMMDA will use for detecting matter. Obtain airborne matter. Collects matter by prop wash and screens. Obtain matter on surfaces using the wiping method with a mechanical arm and fabric Transport matter whereby the UMMDA drones can be equipped with a mechanical arm that will transport matter or deposit it into the reservoir. Repair. If a problem such as a leak, clog, or mechanical breakdown exists with the UMMDA, the drone through a camera and mechanical arm may be able to correct the issue through a technician operating the drone by remote control. The apparatus also has the option to use the drone assisting robots to repair an issue with the apparatus.

Robots as a component of the UMMDA have different methods of operation: Map a 3D view of the area that UMMDA will use for detecting matter.

Robots obtain matter from surfaces on the ground by the wiping of fabric method, dabbing, or dragging. Robots can also transport matter and deposit it into a reservoir or, the UMMDA robot can be equipped with a mechanical arm that will transport matter identified as IDK to the nearest lab for further evaluation. The Robot may also transfer the matter to the UMMDA drone for further transport.

If a problem such as a leak, clog, or mechanical breakdown exists in the apparatus, the robot through a camera and mechanical arm may be able to correct the issue through a technician operating the robot by remote control in a faraway location. The apparatus also has the option to use the robot assisting drones to repair an issue with the apparatus.

Robots may use other methods of gathering matter by rolling spheres on surfaces. Spheres made of different materials of layers of spheres are set inside each other whereby each sphere is hollow and the closer to the center of the sphere, the smaller the sphere is. There can be several layers of spheres whereby the surface area of each sphere contains holes. The rolling of the sphere application on a surface will collect matter that will stick to the surfaces of the sphere. The spheres can then be submerged in the reservoir below the water surface by mechanical arms. Some of the matter on the surfaces of the hollow spheres will be released into the reservoir for detection purposes.

Watercraft as a component of the UMMDA has different methods of operation: Map a 3D view of the area that UMMDA will use for detecting matter. The UMMDA can be placed on top of a watercraft for operation. Pumps can be placed on the watercraft to pump liquid from a body of water into a reservoir located on top of a boat.

The UMMDA watercraft can collect matter from surfaces of water by utilizing a mechanical arm with a cup to retrieve and pour water into a UMMDA reservoir either located at the water's edge or on top of a boat or ship. Watercraft can also be airboats.

Mechanical arms can be stationary, mobile attached to a UMMDA or affixed to the apparatus. They can also be operated remotely, manually, or automated and operated by the UMMDA. For purposes of this patent application, a mechanical arm is defined as a large or small (nanotech-sized) arm that copies the action of a human arm with fingers. Mechanical arm and robotic arm are interchanged in this patent application whereby they can be operated by the apparatus (automated) or by the user (manually) hereafter referred to as a mechanical arm.

The mechanical arm has many uses in this application.

They gather, capture, transport, and separate matter in this patent application. They deposit, drop, throw, dab for matter, drag for matter, use objects and fabrics to obtain matter for detection, or submerge themselves in receptacles by the UMMDA or a user. They submerge an object that may have a matter on its surface into the top of a reservoir. The object once submerged in the liquid of the reservoir will release some of the matter attached to its surface into the circulating water by the blender. The mechanical arm can either submerge its mechanical arm or an object that has matter attached to its surface held by the mechanical arm.

Mechanical arms may be affixed anywhere on the apparatus, next to the matter deposit portal, on drones, robots, and watercraft that may be needed for related issues with other UMMDA mobile vehicles. The mechanical arms use the wiping method with fabric to collect matter for deposit into reservoirs.

A sticky substance receptacle is used for applying a sticky substance to a surface of an object or fabric to attract, capture and maintain matter on the surface of an object whereby the object is submerged (slightly, fully, or anywhere in between) in the sticky substance receptacle.

For purposes of this patent application, a receptacle will be defined as a container that holds either a sticky substance of solid, gooey, or liquid or any other type of container of any material that can hold a liquid, a solid, or a mixture of both whereby a top can be affixed to and the container can be open or closed by a user or can operate mechanically and be automated in operation and does not leak. The purpose of the container is to: allow a user to manually dip an object in the receptacle or allow a UMMDA mechanical arm (just the arm, the arm a claw, or a claw holding an object) to submerge itself below the surface line of the sticky substance such as a liquid or gooey protein. [symbol] A receptacle of sticky substances may be two or more mixed together.

For purposes of this patent application, a soluble substance dissolves in a liquid, usually water. The mixture is a solution that can be transparent. The solid that dissolves is called a solute. Solutions will travel through the liquid tube with ease. Some sticky substances are soluble. The application can also use insoluble substances that only dissolve in liquids other than water. For these, liquid tubes are used instead of the water tube. An example would be if a protein is introduced into a reservoir full of water, the water will be needed to emulsify or release the protein and the matter that has attached to it into the reservoir.

Sticky substances for purposes of this patent application are but are not limited to: silicones starting with PEG, natural hair shampoos, proteins, bacterial secretions, salt, honey, sugar, powdered milk, and cooked rice.

The apparatus reservoir will allow for matter introduced on the surface of an object to be circulated into the liquid tube where large matter such as rice will be filtered out by screens and or filters before it enters the liquid tube.

The method is to allow the sticky substance such as a protein to become coated on a portion of the surface of an object such as a sphere whereby the protein will become stuck to the surface of the sphere after being submerged in a container of protein. Then by the mechanical arm (or by the user), the sphere is rolled across, touched, dabbed, or dragged over a surface whereby matter will be disturbed and the proteins will attract the matter to the surface of the sphere and stick to it until the sphere id submerged by the mechanical arm in the liquid tube reservoir. Since the sticky substance may be water soluble, cleaning the surface with water may be required after capturing matter.

The manual way in which to obtain matter for the manual home application is to use the rolling ball method. The apparatus utilizes spheres that can be coated in a sticky substance such as a protein. The ball can be rolled on surfaces to collect matter by hand. Marking spray paint rolling applicator. The ball has many layers of surfaces with one ball inside another larger ball where many balls can be in one ball.

When a hazardous matter is detected and the UMMDA alert light is blinking, the area may be quarantined. Mechanical arms may be needed on UMMDA mobile vehicles to close off a liquid tube or transfer hazardous matter. Gates may be equipped with some UMMDAs whereby gates internally in liquid tubes will be needed to seal off a biological germ or pathogen that has been detected. Gates are a UMMDA option with specific algorithms whereby law enforcement and military applications will utilize this option.

[symbol]

In a high-end apparatus versions, an expensive version of the apparatus will have adjustable nano filter screens to trap singular forms of very small matter such as viruses. The nano adjustable filter screens “NAF screens” will be embedded in the liquid tubes and operated manually or automatically by the apparatus. The NAF screens will be assisted by nano-mechanical arms that will place the small matter such as viruses directly under the lens for viewing or be placed on top of a cantilever beam for acquiring the weight of the matter.

The distance of the spaces in the screen can be adjusted to only allow microbes the size of viruses and anything smaller the space. The bars of the screen can be adjusted to allow matter as small as 0.22 nanometers whereas a virus can be about 20 nanometers and 400 nanometers in size. Bacteria are about 1 to 2 microns in diameter and 5 to 10 microns long and can also get caught in a screen that is a size a little larger than the bacteria as determined by the apparatus.

An option for the user is to not utilize image enlargement devices and attach lasers that are connected to a separate computer processing device that is programmed with detection algorithms with imaging feeds. The laser(s) can be attached directly above the reservoir or directly above the liquid tube. Lasers can be in the form of solid-state lasers, gas lasers, liquid lasers, chemical lasers, and metal vapor lasers and they have different colors such as green (brighter than a red laser) and red lasers. In this application, lasers are used to detect viruses and bacteria whereby the lasers are affixed to the UMMDA to detect the molecular makeup of microbes in the liquid tube and the reservoir. In this particular user option (usually for research facilities, a gain of function), the level of liquid in the reservoir is very low. Using Raman spectroscopy, the lasers measure the photons of the matter in the reservoir. How the laser detects the type and makeup of matter by directing a laser light down into the reservoir or liquid tube. Other laser user options are to use the embedded microscope slides in the tube, utilize a topper slide or remove the slides from the liquid tube and have the laser point directly into the tube to detect the molecular makeup of the matter in the tube. The lasers may also be set above very thin microscope glass slides for the same type of detection as the UMMDA liquid tube option with embedded slides in the liquid tube.

The specific laser algorithms detect and identify bacteria and viruses and report in real-time.

The lasers help to do two things: help with training algorithms, and detection of matter. The next section is about the power sources for the apparatus.

The power of the apparatus is provided by any one single source or combination of power sources listed below. The power source(s) to the apparatus can be any one or combination of the following power sources consisting of hydrogen fuel cells, nuclear power (nuclear fission or fusion, fusion or fusion of atoms, and fusion energy), combustible engines (using natural gas, gasoline, diesel fuel, petroleum oil based fuels), solar power generated from solar panels, wind power generated from wind turbines, water power generated from water wheels, battery power, magnetics, heat or electricity (or electricity generated from the power sources listed above) or direct wireless charging systems “DWCS”. The applicant has described in prior applications with the USPTO how DWCS work and the methods used. DWCS charging pads are used on top of reservoirs to charge drones, on the side of reservoirs to charge robots, and at the edge of the water to charge watercraft.

Power transfer and creating power for the apparatus through a direct wireless charging system hereafter known as “DWCS”. For purposes of this patent application, Direct Wireless Charging Systems “DWCS” will be defined as pads, rails, trusses, and wires “dwcs” charging devices” that can provide an instantaneous charge to a UMMDA mobile vehicle that maintains a storage device that holds a charge while the UMMDA mobile vehicle is in motion or static. This method is to charge devices without either components having physical contact or being wired physically to a charging device that can hold a charge. The charge can be transferred wirelessly to the UMMDA mobile vehicle while the UMMDA mobile vehicle is close to the dwcs charging device. The apparatus or a component thereof can be charged by replacing a charged device that was charged by the dwcs.

This system works where dwcs charging devices will be laid on the ground, attached to the ceilings and walls, on top or at the edge of the water (the devices are waterproof) where the drones and robots, and watercraft can be charged and power transferred to another component of the apparatus. The dwcs charging devices are plugged into electrical outlets whereby the pads transfer the charge to UMMDA mobile vehicles. The dwcs charging devices can also be charged by any power source listed above such as combustible engines.

The physical aspect of the apparatus has 4 main components: Steel frame, Power generation, reservoirs and auxiliary reservoir components, and Wired and wireless components (male and female connections) Mobile vehicles are not included in this section and are not a requirement for manual home applications.

The steel frame or frames within the steel frame are constructed to hold the apparatus components. They have listed below in no particular order of importance: Laptops, image enlargement devices, electronic circuit boards, and panels for automatic and manual shutdown upon malfunctions, hacks, and sabotage) power cords, wireless, wired components, tubes, CPUs (GPUs), laptops, cell phones, tablets, servers satellite equipment, CB radio (citizens band radio) and their connections, wireless communication equipment, wired male and female connections, cords, lights (to light up the apparatus, microscope slides, for communication by light, for surrounding area of apparatus, tablets, servers, power connection (or just one main connection for entire apparatus shutdown and components) power generation device frames for stability, antennas,

For ease of setup and breakdown of the apparatus, there are high-end versions of the apparatus and manually operated versions of the apparatus that are low-end. Each version has a different setup and breakdown whereas the lower-end version of the apparatus has a lot fewer components than the higher end. For purposes of this patent application, the high end would be defined as the version of the apparatus being equipped with most components discussed in this patent application which includes all types of computer software programs and algorithms discussed in this patent application. The lower-end version has limited components as discussed in this patent application with little to no computer software programs or algorithms. The lowest-end version of the apparatus will have real-time detection and identification algorithms, a laptop, tube (two embedded slides, 1 pump). The next version up will start to include more and more components. With the viewing algorithms included, this is where the next version is available. The higher-end options become available and with the higher-end apparatus versions, choosing from the list of components listed in this patent application are available.

The low end of the apparatus can be transported in a single pelican case while the higher end apparatus can be shipped in many pelican cases. For large municipal apparatus detection, identification, and viewing operations, the apparatus will be constructed on-site with some components being shipped in pelican cases.

Setup of apparatus. The setup of the apparatus is determined by the apparatus version. The time needed is determined by many factors with the main factor being the size of the operation. If a manual apparatus is being set up by the user for an in-home operation, the apparatus can be taken out of a pelican case, set on a tabletop, and plugged in. The manual operation of the apparatus can be guided by the instructions. The apparatus can be plugged n used after filling the circulation tank with water.

The user can decide to discard the tube and keep all the other components and purchase new tubes for other operations. The apparatus components can be rented leased, licensed, or purchased where there is no need to break down the apparatus.

The platform of the apparatus trusses, metal frame, wired, wireless, places for laptops on the metal platform of apparatus wires, USB, cat 5, electrical, electrical extension cords.

For transport, set up, and or breakdown of tubes in great length and or sections, the tubes may be designed to be folded, coiled, or wound up for storage in a box.

The first step in setup—is the setup of the tube component. The first option is whether the user desires the manual or automatic version. Components may be wired to the apparatus or connected wirelessly. There is also an option to operate the apparatus manually. of tubes in great length and or sections, the tubes may be designed to be folded, coiled, or wound up for storage in a box. A liquid pump can be used to remove liquids from the tube before the transport tube.

Environmental applications that require little disturbance to the immediate apparatus location or locations miles from where the apparatus is placed and or operational, the apparatus through its algorithms combined with software programs learns what actions to perform and not perform in specific environments. An environmental disturbance will be defined in this patent application as the physical displacement of matter that affects environments in a toxic and unhealthy manner for all living organisms including plants, animals, and human beings.

FIG. 1 is a schematic diagram for the sample reservoir connected to the transparent liquid tube “liquid tube” FIG. 2 according to an embodiment of the present disclosure. FIG. 1 depicts a sample reservoir with an entry point from an open top whereby UMMDA mobile vehicles or a user can place matter, liquids, or micro-organisms through the open top. FIG. 1 also depicts an entry side port in sample reservoir whereby the open end 101 is placed into a body of water. The pump 102 pulls water into the sample reservoir. 103 is the inlet tube into the sample reservoir. 104 is a manual shut off value to hold liquid and matter in the reservoir on the inlet tube. 105 is the connection to the control unit to open and close the valve. 106 is the valve unit. 107 is the overflow petcock. 108 is the blender device with 6 teeth. 109 is power to lower pump whirlpool creator. 110 lower whirlpool pump device. 111 intake and jet stream tube. 112 top tube to whirlpool pump. 113 top whirlpool pump device. 114 is power to top whirlpool pump device. 115 is the connection to the control unit to open and close the outlet tube valve. 116 is the valve unit to the sample reservoir exit tube. 117 is the manual valve shut off to the sample reservoir exit tube. 118 is the sample reservoir exit tube. 119 is the open-end exit point that is either placed in a body of water or directed elsewhere.

FIG. 2 is a schematic diagram for the transparent liquid tube according to an embodiment of the present disclosure. FIG. 2 depicts a CPU 205, a power switch 208 for apparatus which includes both the liquid tube and the CPU which may be a server, a laptop, a tablet or a cell phone; power station and connections 209 for liquid tube lights, adjustable microscope slides, microscopes and mobile components and data transmission, wired and wireless; encased wire and cords 203 for power, wired data connections; laptop or CPU connected by wire; a bottom wall 105 of transparent liquid tube; an outlet 207; an inlet 201; and encased wire and cords 212 for power and wires that connects top and bottom encased wired connections. Wire is outside of liquid tube; encased wire and cords 204 for power, wired, wireless data connections, auxiliary connections; a component and auxiliary connection box 210 for mobile and wireless components; power cord 211 for electric, wherein the power cord 211 is electric where any type of power can be converted to electric power and plugged in; and a top wall 202 of transparent liquid tube.

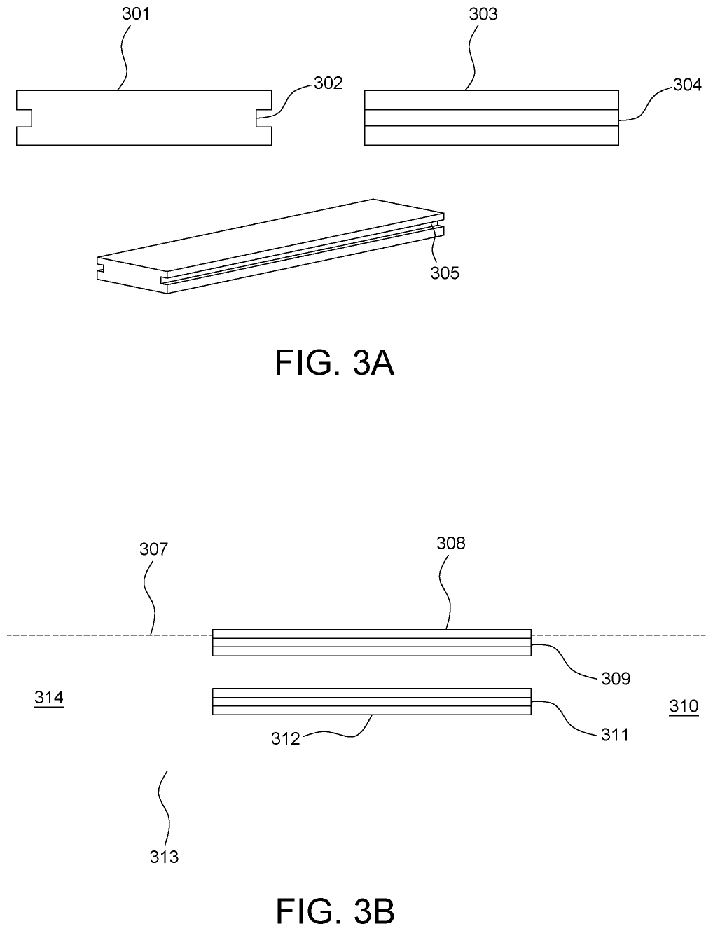

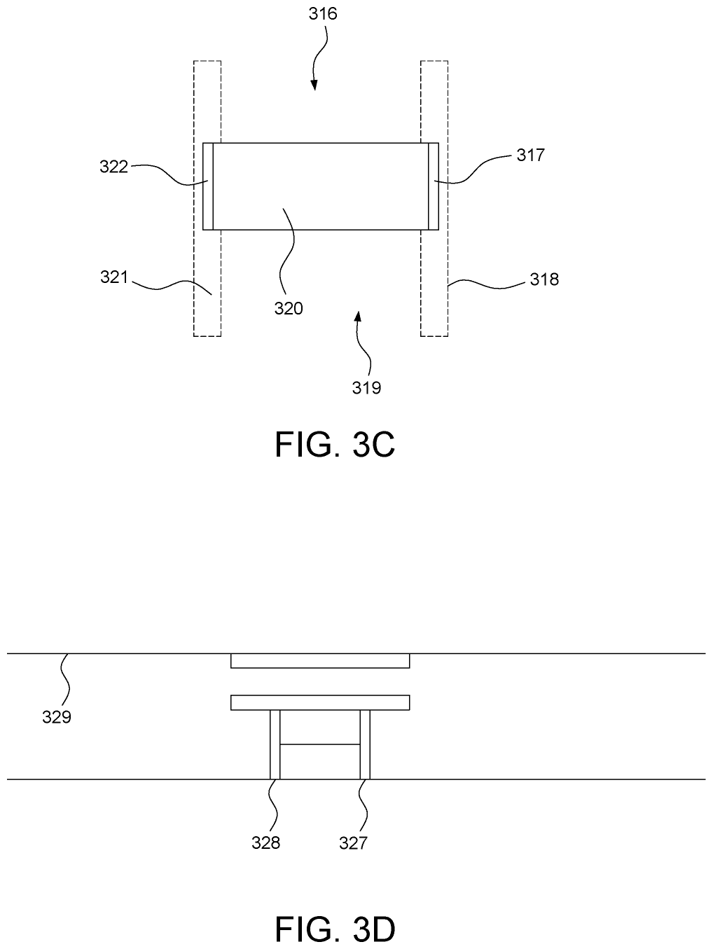

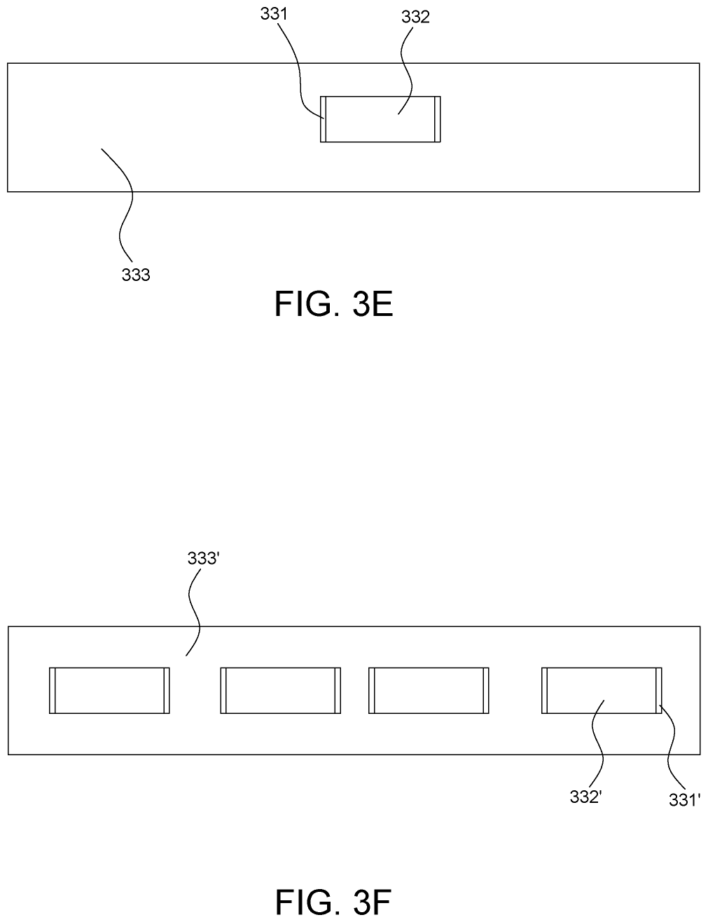

FIGS. 3A to 3F are schematic diagrams for the detection unit of the transparent liquid tube. FIG. 3A depicts a front view 301 of microscope slide; a front view of microscope slide with H groove for holding slide embedded in liquid tube—302; a side view 303 of microscope slide with H groove; 304 a side view of H groove and a 3-dimensional view of embedded microscope slides 305. FIG. 3B depicts a top view 307 of transparent liquid tube “TLT”; a top microscope slide 308 embedded in Transparent Liquid Tube “TLT”; a H groove 309 in side of top microscope slide; a side view 310 of TLT right side of tube open; a H groove 311 in side of bottom microscope slide; a bottom microscope slide 312 embedded in the side of the TLT; a bottom wall of transparent liquid tube “TLT” 313; and a side view 314 of left side of open tube. FIG. 3C depicts a perspective view 316 of the transparent liquid tube left side view of top microscope slide. FIG. 317 is right end of microscope slide with H groove. FIG. 321 is the left wall of the liquid tube. FIG. 318 is the right wall of liquid tube. FIG. 321 is the left wall of liquid tube. FIG. 322 is the left end of microscope slide with H groove. FIG. 320 is a top view of top slide. FIG. 319 is the flow of matter and liquid over surface of microscope slide. FIG. 3D depicts a right brace 327 internally for bottom microscope slide; a left brace 328 internally for bottom microscope slide. FIG. 329 is a top wall of liquid tube with embedded top microscope slide in wall of liquid tube. The left side and right side of liquid tube are both open for input and output of liquid and matter. FIGS. 3E and 3F depict a top view of top microscope slide FIG. 332, with H groove FIG. 331; a top view of top slide of TLT FIG. 333. FIG. 3F depicts 4 microscope slides in liquid tube.

The present disclosure provides an apparatus and method for detecting one or more of a matter and a plurality of micro-organisms. The apparatus includes a plurality of liquid tubes; a plurality of microscope slides; an oil immersion section; a plurality of image enlargement devices; a plurality of remotely controlled unmanned land, air, and water self-propelled devices; a plurality of software program computing systems; a plurality of liquid and air pumps; a plurality of lasers and sensors; a plurality of lasers and sensors; and one or more processors. The microscope slides are embedded in the liquid tubes. The oil immersion section is placed on top of the liquid tubes attached to a plurality of reservoirs. The image enlargement devices are placed on or near the liquid tubes, wherein the image enlargement devices are operated manually, automatically, mechanically, or electronically to enlarge the matter, and the micro-organisms. The remotely controlled unmanned land, air, and water self-propelled devices collect matter and micro-organisms. The software program computing systems utilize software algorithms and software programs written in a plurality of software languages to automatically operate the apparatus and the remotely controlled unmanned land, air, and water self-propelled devices. The software program computing systems direct the elimination of matter and micro-organisms. The liquid and air pumps are controlled by the software program computing systems. The lasers and sensors are controlled by the software program computing systems. The processors execute a plurality of machine learning and artificial intelligence software program algorithms to detect, view and eliminate matter and micro-organisms.

In an embodiment, the apparatus includes a plurality of detecting devices and a plurality of computer software programs for detecting matter in real-time. In an embodiment, the matter comprising biological germs, viruses, bacteria, fungus, protozoa, molds, allergens, disease-forming microorganisms (pathogens), non-disease forming micro-organisms (non-pathogenic), microbes, clusters of micro-organisms, a cluster of matter, hydrocarbons, metals, oils, human and animal bodily fluids, plant matter, fertilizers, chemicals, contaminants and algae in a liquid/wet and/or dry/pseudo-dry liquid tube.

In an embodiment, the apparatus includes a plurality of external and internal lights. In an embodiment, the microscope slides are adjustable manually or by the computer software programs. In an embodiment, the apparatus includes a plurality of direct wireless charging systems to power the components of the apparatus. In an embodiment, the apparatus includes a plurality of direct wireless charging systems to transfer charge to a plurality of other devices in the apparatus. In an embodiment, the apparatus includes a plurality of power devices comprising a battery, nuclear power, natural gas, gasoline and diesel combustible engines, water wheel power, solar panels, wind turbines, and magnetic energy.

In an embodiment, the remotely controlled unmanned land, air, and water self-propelled devices comprise a plurality of mechanical arms to collect, deposit, move, retrieve, and transport matter and micro-organisms. In an embodiment, the mechanical arms are static, mobile, adjustable, and movable, with pinchers. In an embodiment, the image enlargement devices are a plurality of single components and entire light components of a plurality of microscopes. In an embodiment, the plurality of microscope slides are spaced opposite from each other, wherein both liquid and matter travel through space between the microscope slides, wherein the microscope slides embedded in the liquid tubes maintain a surface for receiving and holding liquid and matter. In an embodiment, the microscopes magnify the liquid sample on a sample surface of the microscope slides or between the microscope slides. In an embodiment, the reservoirs are connected to the liquid tubes for holding liquid and matter samples. In an embodiment, the apparatus includes a plurality of inlets for introducing liquid and matter into the reservoirs and the liquid tubes.

In an embodiment, the apparatus includes a plurality of outlets for withdrawing liquid and matter from the reservoirs and the liquid tubes. In an embodiment, the apparatus includes a plurality of light sources emitting light into the liquid tubes and reservoirs. In an embodiment, the apparatus includes a plurality of photodetectors for detecting light transmitted through the liquid tubes. In an embodiment, the apparatus includes a plurality of control units for controlling the operation of the liquid tubes.

In an embodiment, the control units comprise a processor for analyzing the light detected by the photodetectors to determine the presence of the matter in the liquid tubes using a plurality of artificial intelligence learning platforms, the computer software algorithms, and the computer language software programs based on the amount of the light detected.

In an embodiment, the plurality of liquid and air pumps comprise a plurality of processors for monitoring, starting, and stopping the flow of liquid in the liquid tubes using artificial intelligence learning platforms, algorithms, and computer language software programs.

In an embodiment, the sample surface of each microscope slide is comprised of a material selected from a group consisting of glass, plastics, silicone, metals, and combinations thereof. In an embodiment, the plurality of microscopes are configured to provide an image of the liquid sample and the matter sample on the surface of the microscope slides. In an embodiment, the plurality of microscopes are configured to detect fluorescence emitted from liquid and matter on the sample surface of the microscope slides. In an embodiment, the plurality of microscopes are configured to generate a signal indicative of the fluorescence emitted from the liquid sample and matter sample on the sample surface of the microscope slides.

In an embodiment, the plurality of microscopes are fluorescence microscopes. In an embodiment, the plurality of microscopes are light microscopes. In an embodiment, the plurality of microscopes are super-resolution microscopes. In an embodiment, the plurality of microscopes are configured to control the intensity and duration of the light source used to illuminate the liquid sample on the sample surface of the microscope slides.

In an embodiment, the plurality of microscopes are configured to capture the image of the matter on the sample surface of the microscope slides. In an embodiment, the plurality of microscopes are configured to store the captured image from the surface of the microscope slides in a memory device. In an embodiment, the apparatus includes a liquid with matter sample collection receptacle for collecting the liquid sample with the matter sample.

In an embodiment, the apparatus includes a display unit for displaying the results of the matter and micro-organism detection. In an embodiment, the apparatus includes a communication unit for transmitting the results of the matter and micro-organism detection to a remote device. In an embodiment, the battery powers the apparatus and a plurality of components of the apparatus. In an embodiment, the computer software programs automatically reroute matter into the reservoirs connected to the liquid tubes to break apart the matter. In an embodiment, the artificial Intelligence and machine learning algorithms in conjunction with the computing software programs determine a plurality of operations of the apparatus and learn from the operations. In an embodiment, the liquid tubes are connected to a conveyor belt partly submerged in water.

While embodiments of the present disclosure have been illustrated and described, it will be clear that the disclosure is not limited to these embodiments only. Numerous modifications, changes, variations, substitutions, and equivalents will be apparent to those skilled in the art, without departing from the scope of the disclosure, as described in the claims.

One aspect of the present disclosure is to provide a liquid tube which may be transparent on some versions of the apparatus for users to view the apparatus working. The apparatus can be programmed to decrease the accuracy of detection or increase the accuracy by 3 methods. The accuracy can be programmed to be between 55% and 89% accurate. The higher end accuracy (over 75%) is done by software programming. The next option is to slow down the intermittent pump. On higher end versions, the apparatus can maintain numerous image enlargement devices and lengthen the liquid tube to accommodate the devices. This will also increase the detection of pathogens or contaminants in real-time. With this higher end version, motility and mobility of micro-organisms can be detected whereby more data can detect movement. The transparent liquid tube may include a sample reservoir having an entry port and an exit port for holding a liquid sample in pure water as opposed to water that is from the sink in a home. Sometimes, sink water will maintain impurities that show up during detection processes. A primary liquid tube can be transparent having a detection unit embedded therein for detecting the presence of pathogens or contaminants in the liquid sample, a transparent sample inlet, and a transparent sample outlet, wherein the sample inlet is connected to an exit port of the sample reservoir; an auxiliary transparent liquid tube connected to the sample outlet of the primary transparent liquid tube, having a detection unit embedded therein for further analysis of the liquid sample feeding from the primary transparent liquid tube; and a control unit in electronic communication with the reservoir, the primary transparent liquid tube, and the auxiliary transparent liquid tube using artificial intelligence and machine learning platforms.

In some embodiments, the auxiliary transparent liquid tube can be operated when a pathogenic microorganism is initially detected through the primary transparent liquid tube.

In some embodiments, the tube being transparent can show operation speed, clogging and how dirty the water is inside the tube if water is used.

In some embodiments, the apparatus liquid tube can use a liquid other than water.

In some embodiments, alcohol-based liquids are used to kill microbes and show only contaminates.

In some embodiments, the transparent liquid channel may further comprise a display unit for displaying the pathogen detection results.

Referring to FIG. 1, FIG. 1 schematically shows a sample reservoir connected to FIG. 2 a liquid tube according to an embodiment of the present disclosure. The sample reservoir FIG. 1 is connected to a transparent liquid tube having a detection unit. The sample reservoir comprises an entry port and exit port, a pump for pumping liquid samples from the sample reservoir FIG. 1 to the transparent liquid tube (FIG. 2), and an internal blender 108 located at the bottom of the reservoir and having teeth for breaking down solid matter or clusters of microorganisms and generating a whirlpool effect, thereby ensuring efficient processing and mixing the liquid samples in the reservoir to fit between microslides.

The sample reservoir can be defined as a watertight receptacle designed to accommodate a diverse range of matter, including liquids and solids. Additionally, a replaceable filter can be incorporated between the reservoir and the transparent liquid tube, streamlining the filtration process. The reservoir is adaptable, with options for open, closed, or perpetually open configurations, catering to the diverse requirements of various applications and uses.

In some embodiments, for example, upon the liquid departure from the liquid tube in open applications such as a pond, the fluid may be further propelled or channeled into an additional tube or reservoir, designated as the “Biosurfactant Treatment Reservoir” or “Biosurfactant Treatment Tube.” This specialized receptacle is tasked with the introduction of biosurfactants to the liquid post-detection. The Biosurfactant Treatment reservoir serves as a critical learning environment for the algorithms, enabling them to assimilate invaluable knowledge pertaining to biosurfactants, thereby enhancing the overall performance and adaptability of the system in the detection and treatment of various forms of matter.

The “Biosurfactant Treatment Reservoirs” or “Biosurfactant Treatment Tubes” facilitate the interaction of contaminants with biosurfactants or other liquid applications as they traverse the tube or reservoir. Separate image enlargement devices such as microscopes are strategically positioned above or below these specialized reservoirs or tubes, enabling the contaminate elimination identity algorithms to effectively evaluate the efficacy of biosurfactants and their mixtures with other environmental liquid applications in neutralizing contaminants. This assessment is achieved by comparing the enlarged images of pathogens or contaminants before and after the application of biosurfactants.

In some embodiments, the transparent liquid tube may be connected to multiple reservoirs whenever necessary. The multiple reservoirs can be designed for specific purposes, enabling them to hold a variety of liquids, including biosurfactants and various combinations, ratios of biosurfactants, and other environmentally relevant liquid applications. These specialized reservoirs also have the capability to accommodate different dilutions and temperatures of liquids, allowing for a highly adaptable system that caters to diverse requirements and applications. By offering such versatility, these reservoirs play a crucial role in optimizing the overall functionality and efficiency of the present disclosure.

The array of reservoirs containing biosurfactant glycolipids encompasses a diverse range of categories, including microbial biosurfactants, polymeric microbial surfactants, and enzymatically synthesized surfactants. These reservoirs feature an extensive list of glycolipids, which include, but are not limited to, for example, surfactin, iturin, fengycin, lichenysin, serrawettin, phospholipids, rhamnolipid, sophorolipid, trehalolipid, mannosylerythritol-lipids, cellobiolipids, lipoproteins, rubiwettins, trehalose, ornithin, pentasaccharide lipids, viscosin, bacitracin, lipopeptides, or combinations thereof.

Referring to FIGS. 2 to 4, the transparent liquid tube (FIG. 2) comprises at least one tube having a top wall 202 and a bottom wall 105 (see also FIG. 3B), a sample inlet 201 and a sample outlet 207, and a detection unit (see also FIG. 3B) embedded in the tube. The transparent liquid tube can be attached to or electronically or wirelessly communicated with a control unit 205.

Each tube is equipped with shut-off valves that can be turned manually by hand (on all versions of the apparatus) at each end of the tube.

In some embodiments, the tube may have adjustable nano filter screens to trap singular forms of very small matter such as viruses. The nano adjustable filter screens (“NAF screens”) may be embedded in the liquid tubes and operated manually or automatically by the apparatus. The NAF screens can be assisted by nano-mechanical arms that place the small matter such as viruses directly under the lens for viewing or be placed on top of a cantilever beam for acquiring the weight of the matter.

The distance of the spaces in the screen can be adjusted to only allow microbes having size of viruses and others smaller than the spaces. The bars of the screen can be adjusted to allow matter as small as 0.22 nanometers where a virus can be about 20 nanometers 400 nanometers in size. Bacteria are about 1 to 2 microns in diameter and 5 to 10 microns long and can also get caught in a screen that is a size a little larger than the bacteria as determined by the apparatus.

In some embodiments, the sample reservoir FIG. 1. is used to hold a liquid sample. The entry port 101 is used to introduce the liquid sample into the sample reservoir, and the exit port 103 is used to withdraw the liquid sample from the sample reservoir FIG. 1. into the transparent liquid tube FIG. 2. Large liquid samples can be broken apart by the blender 108 so that a solid material or a cluster of materials can be small enough to travel through the liquid tube. The sample reservoir FIG. 1. may have a lower pump 110 and a higher pump 113 to circulate the liquid sample from the bottom to the top. The pump 110 can be controlled by the control unit 205 and operated automatically and intermittently by time intervals and by the duration of the entire operation or can shut down and produce a reverse pumping action to flush out large material caught in the transparent liquid tube.

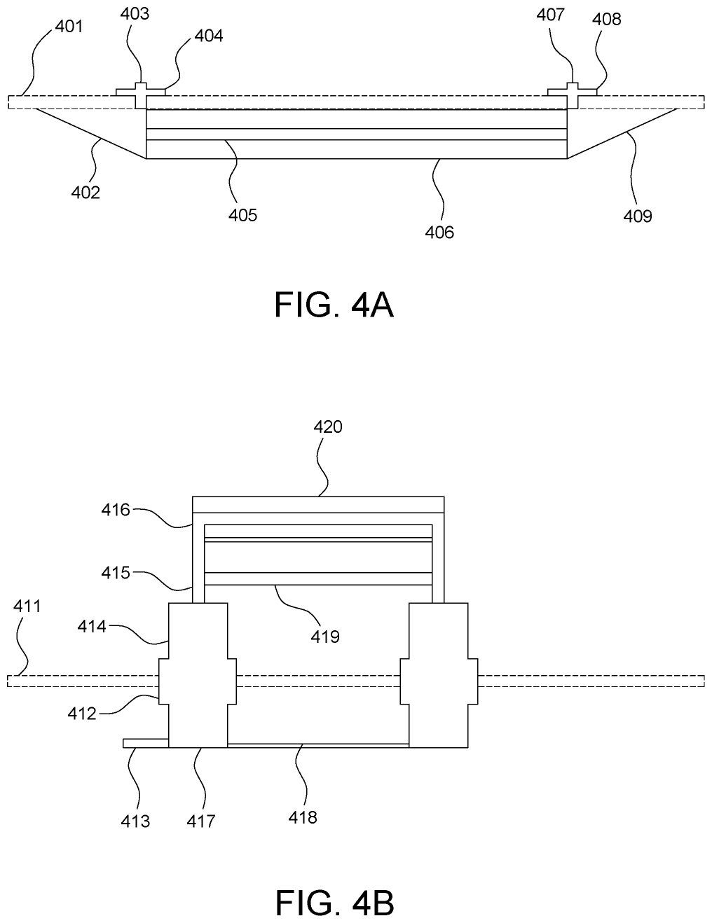

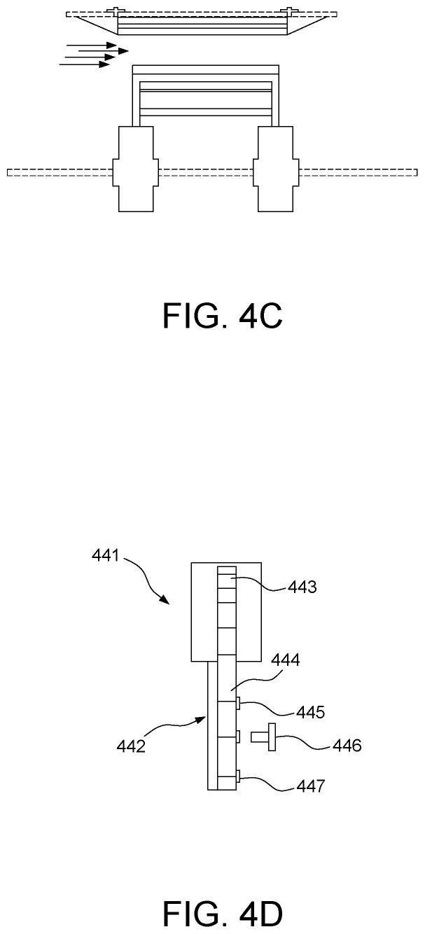

The liquid sample pumped into the transparent liquid tube travels through the detection unit (see FIGS. 4A—top slide and 4B—bottom slide). The detection unit comprises a light source, at least two microslides FIG. 406 and FIG. 420, an image enlargement device located above the top liquid tube wall FIG. 401 (not shown), and a photodetector (not shown). FIG. 406 is the top slide affixed to the top wall of liquid tube. FIG. 420 is the bottom adjustable slide of the liquid tube. The microslides FIG. 406 and FIG. 420 are located in the liquid tube and spaced opposite from each other and have sample surfaces for receiving and holding the liquid sample. FIG. 402 is a slated sectional piece affixed to end of slide FIG. 406. for ease of liquid flow. FIG. 403 is a shaft that goes through the liquid tube wall to hold pin FIG. 404 and FIG. 406 in place. FIG. 405 is an H groove in slide. FIG. 407 is right side shaft to hold the slide. FIG. 408 is the right-side pin. FIG. 409 is the right side slated sectional piece. FIG. 4B is the lower adjustable slide in the liquid tube. FIG. 415 is a horizontal brace for slide. FIG. 419 is the middle horizontal brace and FIG. 416 is an “L” shaped brace. FIG. 411 is the lower wall of the liquid tube. FIG. 414 is the housing of the adjustable piston for lower microscope slide. FIG. 412 is the opening in the housing for the wall of the liquid tube to go through. FIG. 413 is the power cord to the piston device. FIG. 417 is the base for FIG. 418 to hold the right side of housing with no piston device. FIG. 4C shows the direction of the liquid and matter between the top slide FIG. A and bottom slide FIG. B.

In some embodiments, the liquid entering the tube from a reservoir (or the tube itself) may need to be heated or chilled by a cooling or heating element. The chilling or heating can take place internally in the tube, externally surrounding the tube, or upon liquid entering the tube. This may be done by using basic water heating elements such as those in water heaters at homes by electricity, gas or solar power that can be used to change the temperature of the liquid in the liquid tube.

FIG. 3A depicts a dual microslide system, in which two microslides (FIGS. 301, 303) are spatially separated and have grooves (FIGS. 302, 304, 305) located at each side, such that the whole configuration forms an H-shape. These grooves are specifically engineered to securely hold the slides when embedded in the liquid tube (see FIG. 3B), ensuring stability and precise positioning during the analysis process. The depicted arrangement enables simultaneous examination of multiple specimens, facilitating efficient data collection and cross-referencing of results. This microslide configuration not only streamlines the sample analysis workflow but also minimizes the risk of cross-contamination and enhances overall accuracy.

The distance between the microslides within the tube can range from mere nanometers to several inches, offering remarkable flexibility in accommodating diverse applications. For instance, when the microslides are spaced several inches apart, the tube is tailored to address contaminant-related challenges, whereas a smaller gap between the microslides is ideal for targeting viruses. The option provides the ability to manually or automatically adjust the distance between the two microslides, further enhancing the system's adaptability to various scenarios and bolstering its capacity to efficiently tackle an array of environmental issues with unparalleled precision and effectiveness.

When viewing liquid samples through the tube, image enlargement devices such as microscope lenses are employed with or without a condenser, replacing the conventional bottom illuminating light with light sources lining the tube. This approach, known as “running tube lighting or light sources,” illuminates the matter directly beneath the lens for optimal detection. The light sources can be powered by DC, electricity, or various types of lamps, including fluorescent, incandescent, LED, neon, halogen, metal halide, high-intensity discharge, and low or high-pressure sodium lamps. Internally placed lights can be designed as watertight or waterproof to ensure durability.

Further, the light sources may be affixed to the exterior of the transparent liquid tube, providing illumination or functioning as a heat source. In more advanced and costly options, the light sources may be embedded on the sides of one or both microslides or along the interior of the tube. External light sources can also be added to the transparent liquid tube. Such embedded or externally placed light sources may require higher intensity for detecting minuscule matter like parvovirus (20 nm), specific gaseous molecules, or metallurgical matter, necessitating double-layered lighting solutions. Some light sources may also serve the purpose of heating the tube internally, externally, or warming other components of the apparatus, thus enhancing its functionality and adaptability in various applications.

In another embodiment, an option for the user is not to utilize image enlargement devices, but attach lasers that are connected to a separate computer processor that is programmed with detection algorithms with imaging feeds. The laser(s) can be attached directly above the reservoir or directly above the transparent liquid tube. Lasers can be, for example, in the form of solid state laser, gas lasers, liquid lasers, chemical lasers and metal vapor lasers and they have different colors such as green (brighter than a red laser) and red lasers. In this embodiment, lasers are used to detect viruses and bacteria whereby the lasers are affixed to the apparatus to detect the molecular makeup of microbes in the liquid tube and in the reservoir. In this particular user option (usually for research facilities, gain of function), the level of liquid in the reservoir is very low. Using the Raman spectroscopy, lasers measure the photons of the matter in the reservoir. The way in which lasers detect type and makeup of matter is to direct a laser light down into the reservoir or liquid tube. Other laser user options are to use the embedded microslides in the tube, utilize a topper slide or remove the slides from the liquid tube and have the laser point directly into the tube and to detect the molecular makeup of the matter in the tube. The lasers may also be set above thin microscope glass slides for the same type of detection as the transparent liquid tube option with embedded microslides in the liquid tube. The specific laser algorithms detect, identify bacteria and viruses and report in real time. The lasers help to do two things: to help with training algorithms, and to detect matter.

In one embodiment shown in FIG. 3B, a transparent liquid tube incorporates a top microslide (308) and a bottom microslide (312), which are seamlessly embedded within the liquid tube. This allows for precise fluid flow control across the top and bottom microslides (308, 312), enabling real-time observation and analysis of dynamic processes in aquatic environments. The top and/or bottom microslides may be attached to or spaced apart from the wall of the transparent liquid tube. In some embodiments, the top microslide (308) is attached to the top wall (307) of the transparent liquid tube, while the bottom microslide (FIG. 312) is spaced apart from the bottom wall (313) of the transparent liquid tube and can be supported by a right brace (327) and a left brace (328) as depicted in FIG. 3D. The strategic placement of the top and bottom microslides within the transparent or non-transparent “liquid tube” ensures a uniform distribution of the liquid sample, thus providing consistent and reliable detection conditions.

FIGS. 3E and 3F show a transparent liquid tube in which one set or multiple sets of microslides are embedded. These allow for seamless and unobstructed observation of liquid samples under a range of fluid flow conditions. The embedded microslides in FIG. 0.3F are securely positioned within the tube, offering a stable platform for sample analysis, while the transparent nature of the tubing ensures maximum visibility and minimizes optical distortion. The adaptability of this setup to accommodate a series of microslides simultaneously streamlines the detection process and allows for efficient data collection from various samples. Overall, this versatile liquid tube system enhances the precision and accuracy of fluid dynamic detection features.

The sample surface of each microslide (308 and 312) may be composed of a material selected from the group consisting of glass, plastic, silicone, and combinations thereof. The material selected for the sample surface should be transparent and inert, so it does not interfere with detecting any pathogens in the liquid sample. The microslides can be any suitable size and shape. For example, as shown in FIG. 3A, each microslide features an H-shaped groove (302) along its side portion, facilitating easy handling and securing attachment to a holder or other equipment. The microslides can be held in any suitable manner, such as in a holder or a cartridge (327 and 328).

As used herein, the term “microslide” can be used interchangeably with the term “microscope slide” and may have different characteristics when for example the application which can range from a small home to a large airport requires at least one tube having at least 5,000 microslide sets, or more than 150,000 microslides combinations.

In some embodiments, the tube can be equipped with three adjustable microslides options for high end identification and detection of several forms of input large bodies of water, surface matter and matter that is airborne where the apparatus operates on a higher level.

The microslides embedded in the transparent liquid tube are positioned directly below the image enlargement devices (not shown). The image enlargement devices are configured to magnify the liquid sample on the sample surface of the microslides.

There may be many different image enlargement devices the apparatus utilizes. For purposes of understanding the image enlargement device, when the phrase “image enlargement device's is used, it can be any image magnification device listed herein.

The image enlargement devices can be any suitable type of microscopes, and may include, but are not limited to, basic optical lenses, magnification lenses, lenses that are attached to a microscope with a base, folded mirror lenses, light microscopes, electron microscopes, super resolution microscopes, fluorescent microscopes, x-ray machines, magnetic resonance imaging machines, and nuclear magnetic resonance devices, and telescope lenses.

In some embodiments, the image enlargement devices are connected to a computer software device where images and data are transferred wirelessly or by wire.

In some embodiments, the image enlargement devices are placed directly above the slides for detection whereas the devices are connected to computer software and algorithm programs that detect matters in real time.

The image enlargement device defined herein is a mechanically or electronically enlargement measuring device with magnification capabilities. The optical lens enlarges the apparent size (physical size) of matter.

Within the tubes, more than one image enlargement device may not be needed (they may be focused manually or focused by the algorithms of the apparatus). Image enlargement devices that detect mobility, motility, color, size and shape that identifies objects accurately can also be used by the apparatus.

Some image enlargement devices will not utilize microscope condensers. In lieu of the condensers, the tube section underneath (and close by for surrounding light) the image enlargement devices will be embedded with light sources to illuminate the area between the microslides. Some optical lenses each have their own computer software and algorithm programs that manage all the optical lenses throughout the tube internally and externally. At the times when just light sources are used in lieu of the condensers, computer software and algorithm programs will manage the brightness of light required for accurate viewing and detection of matter. The image enlargement devices may be placed externally anywhere outside the tube. The image enlargement devices can be close or far away from the tube. Specific optical magnification lenses may also be placed inside the tube. There is no limit to the number of the image enlargement devices or type of the optical magnification lenses or a combination thereof that can be utilized herein. Algorithms learn from the data obtained from the image enlargement devices and optical magnification lenses and transfer the data to other algorithms in the apparatus. The focusing of the image enlargement devices can be manually operated by a single person or with more than one group of people where each person from the group can manually focus on a single image enlargement device. The focusing of the image enlargement devices may also be operated by optical magnification lens focusing algorithms and third-party software programs and can also be automated. The tube component can communicate with computer software and algorithm programs that manage the entire tube apparatus.

Depending on the length of the tube, there may be many different types of image enlargement devices as listed herein. The image enlargement devices may be located externally outside the tube, located internally—inside the tube. The image enlargement devices (which may include optical magnification lenses and vary to include folded optics and folded mirror lenses) can be on top of the tube, under the tube, on the side of the liquid tube, or on trusses that brace the liquid tube.

In one embodiment, the microscope is a fluorescence microscope and is configured to detect fluorescence emitted from the liquid sample on the sample surface of the microslides. The microscope can be any suitable fluorescence microscope, such as an epifluorescence or confocal microscope. In yet another embodiment, the microscope is further configured to generate a signal indicative of the fluorescence emitted from the liquid sample on the sample surface of the microslides.

In another embodiment, the microscope is further configured to control the intensity and duration of the light source used to illuminate the liquid sample on the sample surface of the microslides using artificial intelligence and machine learning algorithms. This can be accomplished using any suitable mechanism, such as a mechanical shutter or an electronic controller.

In a further embodiment, the microscope is further configured to capture an image of the liquid sample on the sample surface of the microslides. The captured images may be useful in diagnosing the presence of pathogens or contaminants in a liquid sample. The captured images may be stored in any suitable memory device such as a hard drive or a flash drive. The captured images may be transmitted or displayed to a user through a verbal or non-verbal display unit.

Referring to FIGS. 4A to 4C, FIG. 4A depicts a setup where liquid samples flow from left to right, navigating through and between two strategically positioned microslides (406, 420). It is designed to enable real-time detection and identification of various matters or microorganisms present in the samples. In these Figures, an image enlargement device is placed directly above the top microslide (406), capturing and magnifying the images of the microorganisms as they traverse through the liquid medium, thus facilitating detailed analysis and observation.

In FIGS. 4B and 4C, the top microslide is shown securely attached to the top wall of the liquid tube (401), ensuring stability and consistent alignment during the flow of liquid samples. The bottom microslide (420), on the other hand, is deliberately spaced apart from the bottom wall of the liquid tube (411). This configuration maximizes the effective observation area and promotes the even distribution of microorganisms throughout the samples, resulting in more accurate and representative data collection.

FIG. 4C elaborates on the support mechanism for the bottom microslide (420), which is held in place by a vertical static support bar (415) and a horizontal support bar (419). These supports guarantee the stability and precise positioning of the bottom microslide (420) within the liquid tube allowing for optimal detection and analysis of the microorganisms as they pass between the two microslides (406, 420). The combination of these meticulously engineered components contributes to a highly effective and efficient setup, crucial for advancing our understanding of the diverse world of microorganisms.

FIG. 4D illustrates the internal piston to FIG. 414 FIG. 4D is an internal piston (441), featuring built-in ruler lines (443, 444) and three strategically positioned holes (445, 447) for pegs (446). The ruler lines offer precise and accurate measurements of space to facilitate the fine-tuning of piston movement and ensure optimal control over the liquid sample flow space between the slides within the transparent liquid tube. The incorporation of the peg holes allows for easy and secure attachment of the piston to various components of the present apparatus, enhancing stability and adaptability. This multifunctional piston design plays a vital role in maintaining consistent fluid dynamics, ensuring the reliable and accurate detection and analysis of microorganisms in the liquid samples. The integration of these features into the piston contributes significantly to the overall efficiency and functionality of the present apparatus.

As used herein, the microslides can be adjusted whereby the space between the microslides in the liquid tube can be increased or decreased manually by a user or electronically by the apparatus. There are two ways the adjustable microslides work. One is that only one microslide is adjustable which is the bottom slide whereby the top slide in the liquid tube is embedded into the liquid tube. The second way is a layered three microslide system whereby the top slide is static and the bottom two slides are adjustable. Another completely different way is whereby the top embedded microslide is embedded in the liquid tube (not movable) the bottom microslide is adjustable and a topper slide is placed over the top slide on the liquid tube externally (not movable).

According to some embodiments utilizing three stacked microslide methods, the top microslide is on top of a second microslide where the top slide is close to the second microslide in distance. Another third microslide is used in the apparatus where the difference between the second (middle) microslide and the third bottom slide is twice the distance.

According to some embodiments, the liquid tube has pumps that pump liquids into the tube and pump liquids out of the tube. Pumps can also be inside the tube at any point in the tube, placed outside the tube or at any point affixed to the tube. The pumps can pump liquids, air, matter or a combination thereof. The pumps can be nano sized or large industrial scale tubes such as large municipal water pipes. The tube may be connected by other tubes creating a circular flow or the tube may be open at both ends for a continuous flow of liquids like that of an ocean. The pumps must be managed by the apparatus to allow the time change between intermittent pumping to allow for more time for the matter to stay under the lenses and between the microslides for the auto focusing application to focus. There can be many pumps to move liquids through many parallel tubes with many lenses to get more data with less time when time to focus on matter becomes an issue.

In some embodiments, black lights can be affixed on the present disclosure whereby a camera also located thereon can show the user if blood, urine and semen are present on bedding, floors, rugs and walls. These images can be transferred to the control unit whereby the images can be transferred to the laptop via wire, wirelessly or uploaded to a command center for further evaluation.

Although not shown in the figures, the photodetector can be used to detect pathogenic or non-pathogenic microorganisms or other contaminants. If the detection unit typically detects something, the control unit receives data such as imaging feeds from the detection unit and places a colored box around the detected matter, which can be shown in a display unit, and analyzed and identified by an artificial intelligence and machine learning algorithms in the control unit. When pathogenic microorganisms are detected, an alert signal light blinks. The shapes and colors for each type of microorganisms detected such as a rod or a sphere with colored rims are unique to the present apparatus.

Although not shown in the figures, the photodetector can be used to detect pathogenic or non-pathogenic microorganisms or other contaminants. If the detection unit typically detects something, the control unit receives data such as imaging feeds from the detection unit and places a colored box around the detected matter, which can be shown in a display unit, and analyzed and identified by an artificial intelligence and machine learning algorithms in the control unit. When pathogenic microorganisms are detected, an alert signal light blinks. The shapes and colors for each type of microorganisms detected such as a rod or a sphere with colored rims are unique to the present apparatus.

Machine learning algorithms can be trained to detect all types of matter or microorganisms from imaging feeds and classify them as follows (without limitation): all bacteria classes; bacteria singular—spheres, rods, spirals, strings, etc.; bacteria colonies—sphere colonies, rod colonies, spiral colonies, etc.; all virus classes; virus—rod types, crowns (spike), spheres; virus—colonies; all classes of pests, parameciums, algae, molds; all classes of allergens; all classes of contaminants; general singular matter; and general clusters of matter. The labeling of data can be conducted first in singular form and then in cluster form.

Artificial intelligence and machine learning algorithms can be trained to detect all types of matter or microorganisms from imaging feeds and classify them as follows (without limitation): all bacteria classes; bacteria singular—spheres, rods, spirals, strings, etc.; bacteria colonies—sphere colonies, rod colonies, spiral colonies, etc.; all virus classes; virus—rod types, crowns (spike), spheres; virus—colonies; all classes of pests, parameciums, algae, molds; all classes of allergens; all classes of contaminants; general singular matter; and general clusters of matter. The labeling of data can be conducted first in singular form and then in cluster form.

The present system identifies matter by enclosing it within colored squared boxes (with customized colors according to user preference) at level one, and provides corresponding annotations in the form of text adjacent to the box. On the viewing screen, for example, different colored boxes represent various types of matter:
A dark blue box indicates a rod-shaped bacterium.
A blue box signifies a spiral-shaped structure.
A purple box represents a spherical object.
A yellow box denotes a single paramecium.
A yellow box with a red top line indicates more than one paramecium.
A light green box symbolizes a rod-shaped colony.
A light blue box identifies a spiral-shaped colony.
A red box signifies a spherical colony.

The color boxes can be adjusted to represent different types of matter, such as those visible under black light or found in pet excrement. There are hundreds of color combinations that indicate specific matter types, colonies, clusters, and combinations of matter. In level two, matter is identified with pink, red, or purple double-layered boxes and is labeled as “Alert.” This designation is used to draw attention to data that requires special attention or immediate action.

In the event that a biological germ or unknown matter is detected, the apparatus is ingeniously designed to initiate a series of safety measures. It will promptly shut itself down, transmit alerts, and close off tubes to effectively contain the potential threat. The algorithms at work are programmed to activate a pump within an “auxiliary tube” or “secondary tube” (connected to the primary transparent liquid tube viewing the liquid samples) and subsequently redirect the hazardous material into the auxiliary or secondary transparent liquid tube viewing the liquid samples. Here, a distinct image enlargement device and microslides are employed to reevaluate the threat after it has been circulated through a separate reservoir containing a cocktail of biosurfactants.

Further, each tube is equipped with shut-off valves that can be turned manually by hand at each end of the tube. Each tube has its own pumps where the shut-off valve can be turned 90 degrees to allow for the liquid to be pumped in the auxiliary tube. The tube can be removed by hand.

If the system is in alert mode, and the second action taken after pink, purple and red light blinking is to automatically turn the valves and close off the matter. Then the tube may be disconnected from the apparatus by robots for transport by drones. If manually operated, the entire apparatus or just the liquid tube can be transported to a lab for further evaluation.

If the system is in alert mode, and the second action taken after pink, purple and red light blinking is to automatically turn the valves and close off the matter. Then the tube may be disconnected from the apparatus by robots for transport by drones. If manually operated, the entire apparatus or just the liquid tube can be transported to a lab for further evaluation.

Nano mechanical arms that can be affixed to the tube can also be requested by the apparatus to reach in and retrieve the cordoned off matter for transport in a separate closed reservoir or a drone, robot, or watercraft with a closed reservoir for transporting hazardous material.

Following the circulation of the potentially hazardous material through the separate liquid tube (referred to as a second transparent liquid tube with the Biosurfactant option or “Ridcrobe”), the apparatus evaluates the effectiveness of the biosurfactant cocktail in neutralizing the threat. If the biosurfactant mixture successfully dismantles the cell wall of a virus, decomposes bacteria, or disintegrates the contaminant, the primary (or “main”) transparent liquid tube will be reactivated, and the apparatus will seamlessly resume operations.

In some embodiments, the present disclosure is designed for military and law enforcement applications, enabling the gates and auxiliary tubes to close off the primary transparent liquid tube, and effectively isolating the potentially hazardous matter. Moreover, the tube can be detached from the primary liquid tube and transported to a designated location for further evaluation, as required.

Attracting metals to the area below the microscope lenses in the tube can be installed for the purpose of magnets. For contaminate viewing and detection that may involve metals, magnets may be embedded in the tube to attract metals where the magnets will be placed directly below the microscope lenses so the lenses can view the metal particles. The magnets may be placed externally on the tube or internally in the tube. The externally placed magnets may be pulled a short distance from the tube to release the metal particles or dust and the pumps will clear the tube of the metal fragments, if any.

The control unit is equipped with software applications. The software applications are organized into two distinct categories: general software programs and algorithms that control and manage the transparent liquid tube, and specialized software programs and algorithms that are trained or have learned functionalities through artificial intelligence and machine learning processes. With artificial intelligence and machine learning platforms, the exceptional efficacy of the present high-performance liquid tube apparatus is evidenced by its capacity to meticulously amass an extensive array of data points from diverse environmental sources, thereby ensuring the utmost precision in data acquisition. This remarkable accuracy is achieved through the simultaneous analysis of millions of data points, encompassing a wide variety of matter, from pathogens to contaminants, thus facilitating comprehensive and reliable reporting. Central to this cutting-edge technology is the strategic implementation of an extensive liquid tube system, which, when combined with a versatile assortment of image magnification devices such as a microscope, enables the apparatus to seamlessly detect and evaluate millions of data points, effectively revolutionizing the field of data collection and analysis.

The training with artificial intelligence and machine learning algorithms entails the development of computer code tailored to detect specific matter with remarkable granularity, relying on various attributes such as data, imagery, video, color, motility, mobility, shape, size (including circumference and diameter), and weight. Furthermore, the present disclosure elucidates the integration of additional environmental sensors, cantilevers, and lasers as supplementary detection mechanisms, thereby solidifying the apparatus's position at the forefront of cutting-edge technology and bolstering its capabilities in the realm of environmental monitoring and analysis. As for the additional environmental sensors, cantilevers, and lasers, see U.S. patent application Ser. No. 17/879,932, filed on Aug. 3, 2022, titled “Mobile AI, Cantilever, Robot and Drone Applications,” which is incorporated herein by reference in its entirety.

In some embodiments, the present disclosure employs a suite of specialized trained algorithms, capitalizing on the advancements in artificial intelligence and machine learning algorithms to devise a distinct approach for identifying pathogens or contaminants. These algorithms not only focus on detection but also encompass the seamless operation of various interconnected components, such as pumps, detection units, and drones. The apparatus is thereby able to optimize its performance, ensuring precise detection and effective management for all its constituent elements, ultimately revolutionizing the field of environmental monitoring and analysis.

The present disclosure aims to provide a reliable and efficient method for detecting any microorganisms present in liquid samples, regardless of whether it is a disease-causing microorganism or not. As used herein, the expression “detect pathogens” is not limited only to the detection of pathogenic microorganisms such as bacteria, viruses, and fungi, but also it includes the detection of non-pathogenic microorganisms or contaminants that may be present in liquid samples. In this connection, the term “matter” as used herein can be defined as anything that has mass and takes up space, and includes, but is not limited to, biological germs, viruses, bacteria, fungus, protozoa, molds, allergens, disease-forming microorganisms (pathogens), non-disease forming microorganisms (non-pathogens), clusters of microorganisms, hydrocarbons, metals, oils, human and animal bodily fluids, fertilizers, chemicals, contaminants, algae, water, vapors, fluids and solids which broken apart by a blender in the sample reservoir that may continually turn when the system is on. This comprehensive approach to pathogen detection ensures that the present disclosure has broad applicability and can be used in a variety of settings where the presence of any type of microorganisms needs to be detected and/or eliminated.

The control unit comprises a processor for analyzing the light detected by the photodetector to determine the presence of pathogens in the liquid sample using artificial intelligence and machine learning algorithms based on the amount of light detected. The processor can be any suitable computing device, such as a microcontroller, microprocessor, or computer.

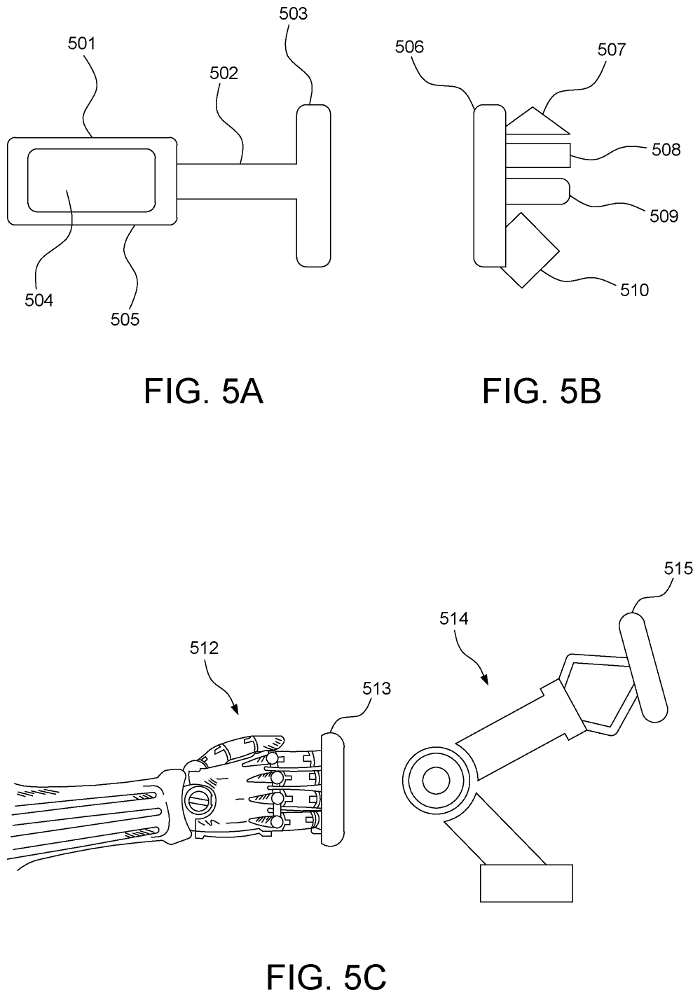

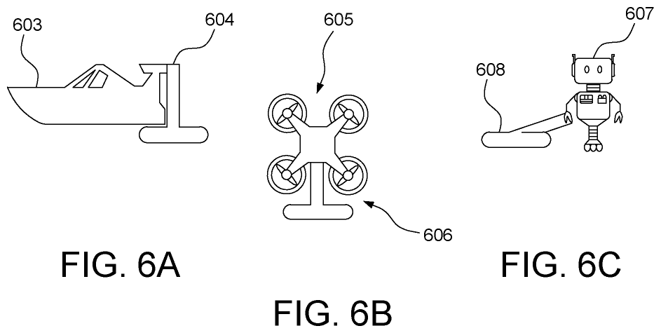

In some embodiments, the liquid tube may further comprise a liquid sample collection unit for collecting liquid samples (see FIGS. 5 and 6). The liquid sample collection unit may be configured to collect the liquid sample from a variety of sources, including, but not limited to, a faucet, a well, body of ocean, brook, lake stream, municipal water treatment plant, a surface, from the air or a natural water source and deposit the liquid samples into the sample reservoir, or a separate or auxiliary reservoir for later evaluation or shipping, connected to the transparent liquid tube. The sample collection unit can be any suitable mechanism for collecting a liquid sample, comprising, for example, a dabbing or dragging device (see FIGS. 5A and 5B), a mechanical arm (see FIG. 5C), a watercraft (see FIG. 6A), a drone (see FIG. 6B), a robot (see FIG. 6C), or any other mobile vehicles with or without a mechanical arm, a pipette, or a syringe.

In some embodiments, the sample collection unit may be a sticky substance as disclosed in U.S. Provisional Application No. 63/345,825, filed on May 25, 2022, entitled ‘Detection All Types of Matter by Spinning Balls with Static Electricity, Sticky Substance on Surface or Magnetized Using Artificial Intelligence,’ which is incorporated herein by reference in its entirety. The method is to allow the sticky substance such as a protein to become coated on a portion of the surface of an object such as a sphere whereby the protein will become stuck to the surface of the sphere after being submerged in a container of protein. Then by mechanical arm or by user, the sphere is rolled across, touched, dabbed or dragged over a surface whereby matter will be disturbed and the proteins will attract the matter to the surface of the sphere and stick to it until the sphere is submerged by a mechanical arm in the sample reservoir. Since the sticky substance may be water soluble, cleaning the surface with water may be required after capturing matter. On the other hand, the manual way in which to obtain matter for the manual home application is to use the rolling ball method. The apparatus utilizes spheres that can be coated in a sticky substance such as a protein. The ball can be rolled on surfaces to collect matter by hand.

In some embodiments, the liquid sample collection unit may comprise a dabbing or dragging device (see FIGS. 5A and 5B). The dabbing or dragging device may have a plastic pole (502, 503, 504) wrapped with linen fabric (501) and/or frayed edge (505), which is a simple and effective tool for collecting liquid samples. The plastic pole provides a sturdy and lightweight handle, while the linen fabric provides a high-quality, absorbent material for collecting liquids. The pole is wrapped tightly with linen fabric to create a smooth, even surface that can be easily dabbed or dragged across a surface to collect a liquid sample. FIG. 5B is another method for collecting matter from surfaces. FIG. 506 is a plastic pole that is affixed to a mechanical arm or a user can hold and utilize to obtain matter from surfaces. FIG. 507 is a solid object in a triangular form to drag along a surface by a user or mechanical arm and then submerge the object in a sample reservoir to release the matter. FIG. 508 is a rectangle shape with a same application as FIG. 507. FIGS. 509 and 510 have the same application but with different shapes that can scape matter from surfaces. FIG. 5C are mechanical arms that hold the dragging and dabbing objects in FIGS. 5A and 5B. FIG. 512 is a mechanical arm that can be attached to the UMMDA or a UMMDA mobile vehicle. FIG. 513 is the section that attaches to FIGS. 5A and 5B. FIG. 514 is another aspect of the mechanical arm. FIG. 515 is the section that attaches to FIGS. 5A and 5B. FIGS. 6A, 6B and 6C are UMMDA mobile vehicles. FIGS. 604, 606 and 608 are the sections that attaches to FIGS. 5A and 5B.

The dabbing or dragging device can be in various shapes, such as a solid triangular shape, solid rectangular shape, or solid rounded rectangular shape. The shape of the device can affect its performance and easy use, with different shapes providing advantages for different types of samples or surfaces. For example, a solid rectangular shape may be more effective for collecting samples from corners or tight spaces, while a solid rounded rectangular shape may be more effective for collecting samples from larger, flatter surfaces. The dabbing or dragging device, including the handle's size and texture and the fabric's absorbency, can also be customized to suit specific applications.

The liquid sample collection unit may be further configured to filter out any debris or other impurities in the liquid sample before introducing it into the sample reservoir. The liquid sample collection unit may be controlled remotely by the control unit.

In some embodiments, the transparent liquid tube may further comprise a communication unit for transmitting pathogen detection results to a remote device. The communication unit may be configured to transmit the pathogen detection results over a wireless communication module, such as Wi-Fi or Bluetooth, or over a wired connection, such as Ethernet or USB. The remote device can be any suitable computing device, such as a smartphone, tablet, or computer.

In yet another embodiment, the liquid tube further comprises a display unit for displaying the pathogen detection results. The display unit can be any suitable type of display, such as a liquid crystal display or an organic light-emitting diode display. In the context of the disclosure, the display unit encompasses a range of display options, including laptop screens, cell phone displays, desktop and server monitors, tablet screens, as well as glass walls and plates that can present data originating from the tube apparatus.

In some embodiments, the transparent liquid tube may further comprise a battery for powering the liquid tube. The battery may be a rechargeable lithium-ion battery or a disposable alkaline battery and may be charged using any suitable charging device, such as a charging dock or a USB charger. The power sources are not limited herein and can be anything conventionally used in the art.

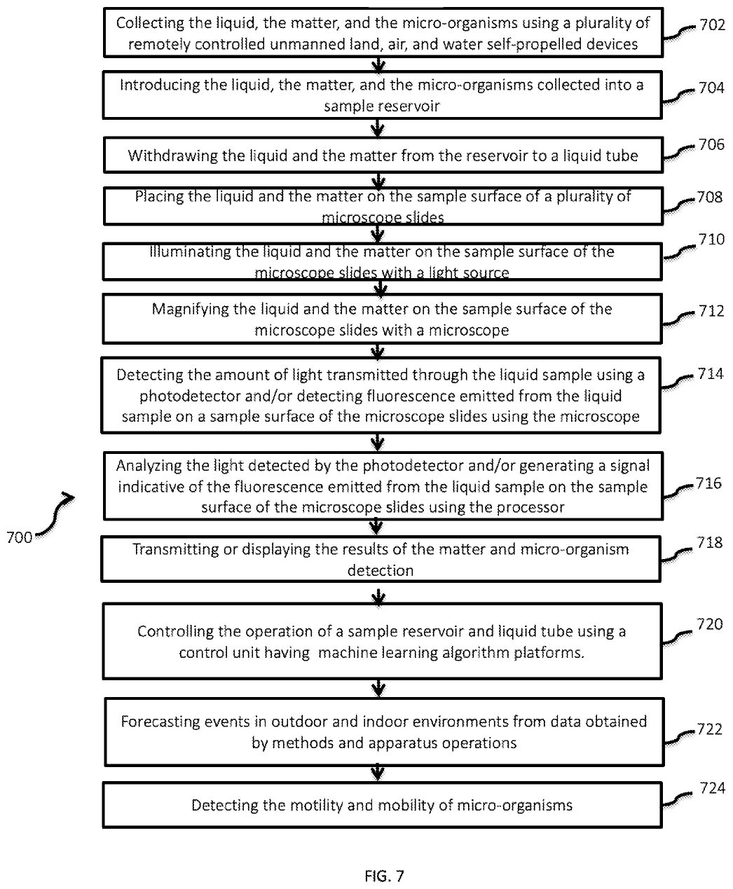

FIG. 7 illustrates a flowchart 700 of a method for detecting one or more of a matter and a plurality of micro-organisms, in accordance with at least one embodiment. The method includes a step 702 of collecting the liquid, the matter, and the micro-organisms in a plurality of remotely controlled unmanned land, air, and water self-propelled devices. The method includes a step 704 of introducing the liquid, the matter, and the micro-organisms collected into a sample reservoir. The method includes a step 706 of withdrawing the liquid and the matter from the reservoir into a liquid tube. The method includes a step 708 of placing the liquid and the matter on the sample surface of a plurality of microscope slides. The method includes a step 710 of illuminating the liquid and the matter on the sample surface of the microscope slides with a light source. The method includes a step 712 of magnifying the liquid and the matter on the sample surface of the microscope slides with a microscope. The method includes a step 714 of detecting the amount of light transmitted through the liquid sample using a photodetector and/or detecting fluorescence emitted from the liquid sample on a sample surface of the microscope slides using the microscope. The method includes a step 716 of analyzing the light detected by the photodetector and/or generating a signal indicative of the fluorescence emitted from the liquid sample on the sample surface of the microscope slides using the processor to determine the presence of matter in the liquid sample. The method includes a step 718 of transmitting or displaying the results of the matter and micro-organism detection. The method includes a step 720 of controlling the operation of a liquid tube using a control unit having a plurality of machine learning algorithm platforms. The method includes a step 722 of forecasting a plurality of events in outdoor and indoor environments from data obtained by methods and apparatus operations. The method includes a step 724 of detecting the motility and mobility of micro-organisms.

Another aspect of the present disclosure is a method of detecting pathogens in a liquid sample. The method for detecting pathogens in a liquid sample involves the use of the transparent liquid tube as described herein. The method begins by introducing the liquid sample into the sample reservoir through the entry port. The liquid sample is then withdrawn into the transparent liquid tube through the exit port. In one embodiment, the pump can be operated by time intervals and by the duration of the entire operation or can shut down and produce a reverse pumping action to flush out large material caught in the liquid tube. This allows for a controlled and precise amount of the liquid sample to be placed onto the sample surface of the microslides, which are specially designed for pathogen or contaminant detection as described herein. The liquid sample pumped from the sample reservoir travels through the liquid tube with the detection unit embedded therein.

After the liquid sample is placed on the sample surface of the microslides while traveling the liquid tube, it is illuminated with a light source or lasers, and the resulting image is magnified using a microscope. The amount of light transmitted through the liquid sample is detected using a photodetector, or alternatively, fluorescence emitted from the liquid sample on the sample surface of the microslides is detected using the microscope.

The light detected by the photodetector and/or the fluorescence emitted from the liquid sample on the sample surface of the microslides is analyzed using a processor, which generates a signal indicative of the presence or absence of pathogens or contaminants in the liquid sample. The signal is then transmitted to the control unit or displayed to the user, using artificial intelligence and machine learning algorithms to identify pathogenic or non-pathogenic microorganisms in the liquid sample accurately. The control unit is designed to analyze large amounts of data quickly and accurately, making it a powerful tool for pathogen or contaminant detection and identification.

The artificial intelligence and machine learning algorithms in the control unit can be trained on large databases of pathogenic and non-pathogenic microorganisms, allowing them to differentiate between the two accurately. The algorithms are designed to adapt and improve over time, learning from each new data point and becoming more accurate with each analysis.

Once the signal is transmitted to the control unit, the algorithms quickly analyze the data and generate a report identifying any pathogenic microorganisms in the liquid sample. The report can be customized to provide detailed information about the type of pathogens, its concentration, weight, and any other relevant information.

By using advanced artificial intelligence and machine learning algorithms, this method of pathogen or contaminant detection provides a high level of accuracy and reliability, making it a valuable tool for a wide range of applications.

In some embodiments, in addition to the steps involved in detecting and identifying pathogens in a liquid sample, the method may further comprise applying biosurfactant to eliminate pathogens or any other contaminants in the environment. Biosurfactants are naturally occurring compounds that have been shown to be effective at breaking down and removing a wide range of contaminants, including bacteria, fungi, and viruses as mentioned herein above.

By applying biosurfactant to the environment, the method can help to reduce the overall pathogen load, making it easier to detect and identify any remaining pathogens in the liquid sample. Biosurfactants work by breaking down the outer membrane of microorganisms, causing them to lose their structural integrity and die off.

In addition to their effectiveness at eliminating pathogens and other contaminants, biosurfactants are also environmentally friendly and non-toxic, making them a safe and sustainable alternative to traditional cleaning and disinfection methods.

Further, another aspect of the present disclosure is a system for detecting pathogens in a liquid sample, which may include the transparent liquid tube and a remote device for receiving the pathogen detection results transmitted from the transparent liquid tube, as described herein. The remote device may be any suitable device capable of receiving and displaying the pathogen detection results, such as a computer, a smartphone, or a tablet.

It should be noted that the design and configuration of the transparent liquid tube may vary depending on the application and specific requirements. For example, the size, shape, and material of the sample reservoir, sample inlet, and sample outlet may be modified to accommodate different types and volumes of liquid samples. Additionally, the liquid sample collection unit may be integrated into different locations of the transparent liquid tube, depending on the space and access requirements.

The present system for detecting pathogens or contaminants in real-time provides an innovative solution to the problem of timely and accurate pathogen or contaminant detection. By incorporating advanced detection technology and an artificial intelligence and machine learning platform, the system can detect pathogens or contaminants in liquid samples with high accuracy and speed. Additionally, including a battery, liquid sample collection unit, display unit, and communication unit in some embodiments of the disclosure makes it a versatile and convenient tool for use in various settings.

The present disclosure described herein should not be limited to the disclosed embodiments but rather includes all modifications and variations that may be implemented. For instance, various types of reservoirs, tubes, channels, and mobile carriers may be used for specific applications. The scope of this disclosure is intended to encompass all such modifications and variations as described in the following claims.

1. A method for detecting one or more of a matter and a plurality of microorganisms, comprising:
a. collecting a sample comprising liquid, matter, and/or microorganisms using a plurality of remotely controlled unmanned land, air, and water self-propelled devices;
b. introducing the sample into a sample reservoir connected to a liquid tube inlet;
c. withdrawing the sample from the sample reservoir into the liquid tube having a plurality microscope slides embedded therein through a sample outlet;
d. placing the sample on a sample surface of the plurality of microscope slides;
e. illuminating the sample on the sample surface of the plurality of microscope slides with a light source;
f. intermittently pumping the sample between the microscope slides from the sample reservoir;
g. magnifying the sample on the sample surface of the microscope slides with an image enlargement device;
h. detecting the amount of light transmitted through the sample using a photodetector and/or detecting fluorescence emitted from the sample on the sample surface of the microscope slides using a microscope;
i. analyzing the light detected by the photodetector and/or generating a signal indicative of the fluorescence emitted from the sample on the sample surface of the microscope slides and transferring that signal to a computer software device to determine the presence of matter and microorganisms in the sample;
j. transmitting or displaying the results of the matter and microorganisms detection;
k. controlling the operation of pumps in the liquid tube using a control unit having a plurality of algorithms that manage the operation and identify and classify the sample;
l. detecting a motility and mobility of microorganisms, color of matter, mass of matter, and type of contaminants or matter, wherein the matter and microorganisms is in a singular or combination form; and
m. forecasting a plurality of events in outdoor and indoor environments from data obtained by step l.

2. The method of claim 1, wherein the plurality of remotely controlled unmanned land, air, and water self-propelled devices are controlled by a control unit.

3. The method of claim 1, wherein the liquid sample is collected from a natural body of water, a man-made body of water, a wastewater treatment plant, or an industrial facility.

4. The method of claim 1, wherein the liquid tube comprises a plurality of interconnected tubing sections that can be adjusted to change the length of the tubing and the position of the sample outlet.

5. The method of claim 4, wherein the plurality of interconnected tubing sections comprise reservoir pump tube, pump exit tube, liquid viewing tube, and reservoir entrance tube.

6. The method of claim 1, wherein the image enlargement device comprises a microscope, a camera, or a combination thereof.

7. The method of claim 1, wherein the image enlargement device is a laser selected from the group consisting of solid state lasers, gas lasers, liquid lasers, chemical lasers, and metal vapor lasers.

8. The method of claim 1, wherein the computer software device comprises one or more algorithms for detecting and identifying the presence of microorganisms or matter in the sample.

9. The method of claim 1, wherein the control unit is programmed with algorithms for optimizing the operation of the pumps in the liquid tube based on the characteristics of the sample.

10. The method of claim 1, wherein the step of forecasting a plurality of events in outdoor and indoor environments comprises predicting the growth of microorganisms, the spread of contaminants or diseases, or changes in environmental conditions based on data obtained from step I.

11. The method of claim 1, further comprising transporting the sample into an auxiliary reservoir and/or an auxiliary liquid tube for further evaluation when an alert mode turns on by detecting pathogenic microorganisms.

12. The method of claim 11, wherein step b through step m are repeated when an alert mode turns on by detecting pathogenic microorganisms.

13. The method of claim 11 or 12, further comprising eliminating pathogenic microorganisms using a biosurfactant, wherein the biosurfactant is selected from the group consisting of surfactin, iturin, fengycin, lichenysin, serrawettin, phospholipids, rhamnolipid, sophorolipid, trehalolipid, mannosylerythritol-lipids, cellobiolipids, lipoproteins, rubiwettins, trehalose, ornithin, pentasaccharide lipids, viscosin, bacitracin, lipopeptides, and combinations thereof.

14. An apparatus for detecting a matter and microorganisms, comprising:
at least one sample reservoir having an sample inlet and a sample outlet for holding a sample comprising liquid, matter, and microorganisms;
at least one liquid tube connected to a sample outlet of the sample reservoir, wherein the liquid tube comprises a plurality of microscope slides embedded therein, a plurality of image enlargement device located above or below the microscope slide, and a light source for emitting the sample on the plurality of microscope slides; and
a control unit having computer software and algorithm programs that manage the operation of the apparatus and identify and classify the sample, wherein the control unit is in electric communication with the at least one sample reservoir and the at least one liquid tube.

15. The apparatus of claim 14, wherein each liquid tube comprises interconnected tubing sections having a reservoir pump tube, a pump exit tube, a liquid viewing tube, and a reservoir entrance tube.

16. The apparatus of claim 14, wherein the microscope slides are adjustable so that a space between the microscope slides in the liquid tube can be increased or decreased.

17. The apparatus of claim 16, wherein one of the microscope slides is fixed and the other of the microscope slides is movable.

18. The apparatus of claim 16, wherein the microscope slides are a layered three-slide system, a top microscope slide being fixed, and a middle and a bottom microscope slide being adjustable.

19. The apparatus of claim 14, wherein the liquid tube comprises a primary liquid tube and an auxiliary liquid tube, wherein the primary liquid tube firstly detect the presence of matter or contaminants in the sample, and the auxiliary liquid tube optionally operates for further evaluation thereof if an alert mode turns on when the matter or contaminants contains pathogenic microorganisms.

20. The apparatus of claim 19, further comprising eliminating the pathogenic microorganisms using a biosurfactant, wherein the biosurfactant is selected from the group consisting of surfactin, iturin, fengycin, lichenysin, serrawettin, phospholipids, rhamnolipid, sophorolipid, trehalolipid, mannosylerythritol-lipids, cellobiolipids, lipoproteins, rubiwettins, trehalose, ornithin, pentasaccharide lipids, viscosin, bacitracin, lipopeptides, and combinations thereof.
